(19) United States
(12) Patent Application Publication
(10) Pub. No.: US 2016/0002664 A1
(43) Pub. Date: Jan. 7, 2016

(54) GENERATION OF CROPS RESISTANT TO CEREAL RUST DISEASE BY SILENCING OF SPECIFIC PATHOGEN GENES

(71) Applicant: WASHINGTON STATE UNIVERSITY, Pullman, WA (US)

(72) Inventors: Scot Howard HULBERT, Pullman, WA (US); Chuntao YIN, Pullman, WA (US); Samantha Llene DOWNEY, Pullman, WA (US); Michael Odell PUMPHREY, Pullman, WA (US); Xianming CHEN, Pullman, WA (US)

(21) Appl. No.: 14/767,611

(22) PCT Filed: Feb. 10, 2014
(86) PCT No.: PCT/US2014/015545

§ 371 Date: Aug. 13, 2015

Related U.S. Application Data
(60) Provisional application No. 61/764,671, filed on Feb. 14, 2013.

(51) Int. Cl. C12N 15/82

(52) U.S. Cl. CPC C12N 15/8282; C12N 15/8218

(57) ABSTRACT

Genetically modified (transgenic) true grasses that are resistant to infection by rust fungi are provided, as are methods of making such transgenic plants. The true grasses are genetically modified by gene silencing of fungal patliogenicity genes that are normally expressed in haustoria.

### BACKGROUND OF THE INVENTION

1. Field of the Invention

The invention generally relates to true grasses that are resistant to rust fungal to infection. In particular, the invention provides genetically modified true grasses in which fungal pathogenicity genes that are normally expressed in haustoria are silenced, rendering the grasses resistant to infection by rust fungi.

2. Background of the Invention

Rust fungi cause devastating diseases of wheat and other cereal species that are the staple food sources in many areas of the world. Three Puccinia species attack wheat; P. graminis f. sp. tritici (Pgt), P. triticina (Pt), and P. striiformis f. sp. tritici (Pst) cause stem rust, leaf rust and stripe rust respectively. The damage caused by each of these rusts has made the development of resistant varieties a high priority for wheat breeding programs around the world. Resistance breeding has been a constant effort due to the ability of these fungi to evolve new pathotypes to which previous resistance is not effective. In the 1990's, wheat breeders and rust workers in the US and most other parts of the world were more focused on finding sources of resistance to leaf rust and stripe rust because sources of resistance to stem rust had been effective for many years. This changed in 1999, when a new highly virulent strain of Pgt, Ug99 (race TTKSK), was identified in Uganda. This and subsequent virulent pathotypes have recently spread into other African countries and the Middle East. Currently, approximately 80% of the wheat cultivars grown in the at-risk areas are susceptible to Ug99. Epidemics of these virulent strains could result in near-total crop loss and are considered a major threat to world food security. The appearance of these new virulent strains to what had been for decades resistant varieties illustrates the urgent need for development of truly durable rust resistance in cereals.

There is a need in the art to develop methods for combating rust fungi in crops. In particular, novel approaches for development of durable resistance to highly variable fungal pathogens are desirable.

### SUMMARY OF THE INVENTION

The genomic sequences of Puccinia species (P. graminis, P. triticina and P. striiformis) have in large part been determined and are available online. However, the biological functions of the individual genes have not hitherto been determined, especially with respect to pathogenicity of the fungi. The present invention provides this information for several Puccinia genes, in particular identifying those haustorial genes which are necessary for fungal colonization and/or reproduction within plants. Haustoria are the fungal cells which reside inside the walls of plant cells, derive nutrients from the plant cells and exchange molecular signals with the plant cells to minimize resistance responses from the plant. Significantly, in spite of the scarcity of genomic tools for rust fungi, rust genomic sequence information has been used to develop viral-based gene silencing constructs and methods for combating rust fungal infection of wheat and other grasses that are susceptible to fungal infections.

Other features and advantages of the present invention will be set forth in the description of invention that follows, and in part will be apparent from the description or may be learned by practice of the invention. The invention will be realized and attained by the compositions and methods particularly pointed out in the written description and claims hereof.

In one aspect, the invention provides constructs comprising one or more P. graminis f. sp. tritici (Pgt) genes may be or include, for example, PGTG_{—}11658, PGTG_{—}01136, PGTG_{—}03590, PGTG_{—}01215, PGTG_{—}03478, PGTG_{—}01304, PGTG_{—}07754, PGTG_{—}12890, PGTG_{—}14350 and PGTG_{—}16914.

In another aspect, the invention provides host plants that are stably transformed to contain and express fragments of one or more P. graminis f. sp. tritici (Pgt) genes selected from the group consisting of PGTG_{—}11658, PGTG_{—}01136, PGTG_{—}03590, PGTG_{—}01215, PGTG_{—}03478, PGTG_01304, PGTG_{—}07754, PGTG_{—}12890, PGTG_{—}14350 and PGTG_{—}16914.

In yet other aspects, the invention provides transgenic plants that are resistant to infection by a rust fungus, wherein expression of one or more pathogenic rust fungal genes is silenced in the transgenic plants. In some aspects, the transgenic plants are a true grass. In other aspects, the true grass is, for example, wheat, barley, sugar cane, or corn. The rust fungus may be a Puccinia species, e.g. a Puccinia fungus such as P. graminis f. sp. tritici (Pgt), P. triticina (Pt), and P. striiformis f. sp. tritici (Pst). Further, the one or more pathogenic rust fungal genes may be or include, for example, P. graminis f. sp. tritici (Pgt) genes PGTG_{—}11658, PGTG_{—}01136, PGTG_{—}03590, PGTG_{—}01215, PGTG_{—}03478, PGTG_{—}01304, PGTG_{—}07754, PGTG_{—}12890, PGTG_{—}14350and PGTG_{—}16914. In some aspects, the transgenic plants are stably resistant to the infection by a rust fungus. In another aspect, the invention provides methods of making a transgenic plant that is resistant to infection by a rust fungus. The methods comprise a step of genetically engineering a plant to contain and express at least one heterologous nucleic acid that, when expressed in said plant, causes silencing of one or more pathogenic rust fungal genes in said plant. In some aspects, the transgenic plant is a true grass, for example, wheat, barley, sugar cane, or corn. In some cases, the rust fungus is a Puccinia species. Exemplary Puccinia species include P. graminis f. sp. tritici (Pgt), P. triticina (Pt), and P. striiformis f. sp. tritici (Pst). The one or more pathogenic rust fungal genes may be, for example, P. graminis f. sp. tritici (Pgt) genes PGTG_{—}11658, PGTG_{—}01136, PGTG_{—}03590, PGTG_{—}01215, PGTG_{—}03478, PGTG_{—}01304, PGTG_{—}07754, PGTG_{—}12890, PGTG_{—}14350 or PGTG_{—}16914. in some aspects, the transgenic plant is stably resistant to infection by the rust fungus.

### BRIEF DESCRIPTION OF THE DRAWINGS

FIG. 1. Photograph of samples A-M showing reduced virulence of stem rust isolate Pgt7A on wheat cultivar McNair 701 through VIGS assays partially silencing specific rust genes. A, McNair701 infected by Pgt7A; B, McNair701 infected by BSMV:MCS, the VIGS construct without any gene fragments; C, McNair701 infected by BSMV:MCS+Pgt7A; D; McNair701 infected by BSMV: PGTG_{—}11658+Pgt7A (also referred to as Pgt-IaaM); E, McNair701 infected by BSMV: PGTG_{—}01136+Pgt7A; F, McNair701 infected by BSMV: PGTG_{—}03590+Pgt7A; G, McNair701 infected by BSMV: PGTG_{—}01304+Pgt7A; H, McNair701 infected by BSMV: PGTG_{—}07754+Pgt7A; 1, McNair701 infected by BSMV: PGTG_{—}12890+Pgt7A; J, McNair701 infected by BSMV: PGTG_{—}14350+Pgt7A; K, McNair701 infected by BSMV: PGTG_{—}01215+Pgt7A; L, McNair701 infected by BSMV: PGTG_{—}03478+Pgt7A; M, Sr31 infected by Pgt7A.

FIG. 2. Photograph of samples A-G showing reduced virulence of stripe rust race Pst78 on wheat cultivar Zak through VIGS assays partially silencing specific rust genes. A, Wheat cultivar Zak infected by Pst78; B, Zak infected by BSMV:MCS, the VIGS construct without any gene fragments; C, Zak infected by BSMV:MCS+Pst78; D; Zak infected by BSMV: PSTG_{—}04507+Pst78; E, Zak infected by BSMV: PSTG_{—}03360+Pst78; F, Zak infected by BSMV: PSTG_{—}04871+Pst78; G, McNair701 infected by BSMV: PGTG_{—}11830+Pₛt78.

FIG. 3. Photograph of samples A-F showing educed virulence of leaf rust on wheat cultivar McNair 701 through VIGS assays partially silencing specific rust genes. A, McNair 701 infected by Pt; B, McNair701 infected by BSMV:MCS; C, McNair701 infected by BSMV:MCS+Pt; D; McNair701 infected by BSMV: PSTG_{—}04507 +Pt; E, McNair701 infected by BSMV: PGTG_{—}03360+Pt; F, McNair701 infected by BSMV: PGTG_{—}04871 Pt.

FIG. 4A-N. Nucleotide sequences of pathogen genes from P. graminis pv. tritici or P. striiformis f. sp. tritici sequences that reduced pathogenicity when they were silenced by VIGS expression in the wheat plant. The sequences are coding sequences from predicted transcripts of the actual genomic sequences. The shaded sequences are those portions of the genes that were used in the silencing constructs. A, PGTG_{—}11658; B, PGTG_{—}01136; C, PGTG_{—}03590; D, PGTG_{—}01215; E, PGTG_{—}03478; F, PGTG_{—}01304; G, PGTG_{—}07754; H, PGTG_{—}12890; 1, PGTG_{—}14350; J, PGTG_{—}16914; K, PSTG_{—}04507; L, PSTG_{—}03360; M, PSTG_{—}04871; N, PSTG_{—}11830.

FIGS. 5A and B. RT-qPCR-based assessment of PGTG_{—}11658 (Pgt-IaaM) gene expression. A, Pgt-IaaM gene expression in different developmental stages in rust. Ured: urediniospores; InfW: infected wheat leaves; and Haust: purified haustoria; B, Pgt-IaaM gene expression after silencing by BSMV-HIGS compared with the BSMV: MCS control. BSMV: MCS: plants infected with BSMV control and Pgt7A; BSMV:Pgt-IaaM: plants infected with the BSMV:Pgt-IaaM and Pgt7A. All data were normalized against the actin gene of Pgt. Standard deviation was calculated from values obtained from three biological replicates.

FIG. 6A-C. Hormone levels in different rust-infected stages in wheat and rust urediniospores. A, Free IAA levels; B, ABA levels; C, trans-Zeatin levels. Healthy wheat 1 is uninfected wheat at the same stage as infected wheat at 2 days post-inoculation (dpi); Healthy wheat 2 is uninfected wheat at the same stage as infected wheat at 4 dpi; Healthy wheat 3 is uninfected wheat at the same stage as infected wheat at 6 dpi. Values are means and SEs of three replicates. Different letters above columns indicate significant differences for hormone levels (p≦0.05; Turkey's HSD test with α=0.05).

FIG. 7A-E. Transgenic Arabidopsis expressing Pgt-IaaM display phenotypes associated with high-auxin content. A, The hypocotyl and roots of five-day-old transgenic seedlings and wild type Cal-0; B, Five-day-old transgenic seedlings display elongated hypocotyls and roots. White bars: wild type; and shaded bars: transgenic plants. Values are means and SDs of three replicates (n>50). Different letters above columns indicate significant differences (p≦0.05; t-test with α=0.05). C, Four-week-old transgenic plants display narrow and downward-curling leaves; D, Seven-week-old transgenic plants; E, RT-PCR analysis of Pgt-IaaM gene expression in Arabidopsis. Col-0: wild type Arabidopsis; and transgenic plant: transgenic Arabidopsis expressing Pgt-IaaM. Similar results were obtained for three independent transgenic lines.

FIGS. 8A and B. Expression of Pgt-IaaM in Arabidopsis accession Col-0 promotes susceptibility to Pseudomonas syringae DC3000. A, Disease symptoms of Pgt-IaaM transgenic plants and Col-0 at 4 days after inoculation with Psi DC3000. 1, Col-0 mock inoculated; 2, Col-0 challenged with Pst DC3000; 3, Transgenic line 1 challenge with Pst DC3000; 4, Transgenic line 2 challenge with Pst DC3000; 5, Transgenic line 3 challenge with Pst DC3000; 6, Transgenic line 1 mock inoculated. B, Growth of Pst DC3000 on transgenic plants and Col-0. White bars represent wild type and shaded bars represent transgenic plants. Different letters above columns indicate significant differences (p≦0.05; t-test with α=0.05).

### DETAILED DESCRIPTION

Haustoria are major sites of molecular communication between rust fungi and their hosts. Rust proteins such as effectors are known to enter the host cell cytoplasm from haustoria. According to the invention, in order to engineer stable rust resistance in plants, information obtained by transiently silencing the expression of a variety of haustoria-specific or selective genes has been used to identify rust genes that are at least associated with, and that may be essential for, pathogenicity of rust fungi. Plants which are susceptible to rust fungi are then stably transformed to inactivate or silence expression of the identified pathogenic rust genes, resulting in genetically modified plants which are stably resistant to the rust fungus. The invention also encompasses methods of making transgenic plants that are resistant to infection by rust fungi.

In order to identify suitable genes for use in the practice of the invention, 1036 fungal genes of interest were initially identified as being preferentially or selectively expressed in haustoria, compared to expression of fungal genes in whole infected leaves (expression was at least 2× higher in haustoria). Of these, 583 genes with clear homologs in three Puccinia rust species, P. graminis f. sp. tritici (Pgt), P. triticina (Pt), and P. striiformis f. sp. tritici (Pst), were identified. These 583 genes had conserved regions with stretches of more than 21 nucleotides of perfect identity among the three rust species, and had no homology with plant sequences. Eighty eight of these 583 genes (or relevant fragments thereof) were individually incorporated into a Barley Stripe Mosaic Virus (BSMV), and the virus was used to infect a true grass of interest (wheat). Infection of a plant by a BSMV results in expression of BSMV gene products, and also of any other sequences that have been inserted into the virus by genetic engineering, e.g. the rust fungal sequences of the 88 genes, or relevant portions thereof.

Plants infected with the genetically engineered BSMV were then infected with rust fungus and the progress of rust infection was monitored. Plants in which rust infection progressed normally were deemed to have been infected with BSMV which did not contain rust gene sequences that interfered with pathogenicity of the rust fungus. However, if symptoms of rust infection were decreased or prevented in a plant, then the plant was deemed to have been infected with a BSMV which contained a rust gene sequence that was necessary (or at least advantageous) for rust fungus pathogenicity. In this manner, rust genes necessary or essential (or at least contributing to or advantageous for) pathogenicity of the fungus were identified for use in the practice of the present invention. The genes were eventually tested against all 3 major types of rust, Pgt, Pt and Pst, and those which were capable of interfering with infection by all three rusts were identified as excellent candidates for use in producing transformed true grasses that are stably resistant to multiple rust species and races.

The following definitions are used throughout:

“Gene silencing” is a term generally used to refer to suppression of expression of a gene. The degree of reduction of expression may be such that expression is completely or only partially abolished.

“BSMV-VIGS” refers to the use of the RNA virus Barley stripe mosaic virus

(BSMV) in transient gene silencing protocols. BSMV is a tripartite (RNA α, RNA β, and RNA γ) positive-sense RNA virus that infects many agriculturally important monocot species including barley, oats, wheat and maize. BSMV is used as a vector for virus-induced gene silencing (VIGS) by exploiting the fact that infection of plants by viruses activates a posttranscriptional gene silencing defense response in infected plants. In VIGS, a short fragment of a transcribed sequence of a targeted gene of interest from a plant or an infectious agent is inserted into a cloned virus genome, and the recombinant virus is then inoculated onto test plants. (Those of skill in the art will recognize that it is necessary to express only part of a gene to silence it. In fact, the virus will not propagate large DNA sequences, as it makes the virus unstable. Larger fragments can be used in stable transgenic plants, or, alternatively, multiple paired gene fragments may be used together for stable transformation; see below). The introduced virus multiplies and triggers, within the plant, posttranscriptional gene silencing of expression of i) viral genes; and ii) genes corresponding to the recombinant targeted gene sequence of interest from the infectious agent that was introduced into the virus. The plant is thus “primed” to silence the authentic targeted gene of interest when it is expressed by an actual infectious agent that later infects the plant. Silencing leads to a reduction in, or in some cases the complete abolition of, function of the targeted gene of interest, which in turn results in phenotype changes (e.g. resistance to pathogen infection, if the targeted gene is related to pathogenicity).

In one variation of BSMV-VIGS, referred to as “BSMV-host-induced gene silencing” (“HIGS”), a fragment of a fungal gene of interest is inserted in the antisense orientation into the RNA γ portion of the BSMV downstream of the stop codon of the γ b open reading frame. As above, the BSMV is used to infect a plant. RNA silencing signals generated against the gene of interest expressed from BSMV within the plant cell can persist and trigger gene silencing in actual fungal cells in intimate contact with the genetically modified host cells. This may occur, for example in fungal haustorial cells which are separated from plant cell membranes by only the extrahaustorial matrix (EHM). The specific mechanisms by which HIGS occurs are not yet known, but, without being bound by theory, it is believed that fungus-specific siRNAs generated by host plant Dicer-like enzyme (DCL) activity may be involved.

“Transformation” or “genetic engineering” refers to the transfer of a foreign polynucleotide sequence into the genome of a host organism such as that of a plant or plant cell. A plant that is “transformed” (i.e. is “transgenic” or “genetically engineered”) is thus one that has been genetically altered by human manipulation e.g. using molecular biology and/or other laboratory techniques to contain one or more nucleic acid sequences that are “foreign” or “non-native” or “heterologous” sequences (e.g. “transgenes”), i.e. sequences that are not found in the plant in nature (are not found in wild-type or control plants of the same species, variety or cultivar). Generally, the foreign nucleic acid is inserted into the plant via techniques, e.g. using a nucleic acid construct or vector (expression vector, expression cassette, plasmid, DNA preparation, etc.) that contains the non-native sequence. Generally, the non-native sequence is located or positioned within the transformed plant so that it is operably linked to (under transcriptional control of) other sequence elements which promote transcription of the foreign nucleic acid within the transgenic host plant, e.g. promoters, and enhancer sequences, along with sequences to terminate transcription. The elements promote constitutive transcription (in all cell types) or promote transcription in more specific cell types, like leaf cells. In some embodiments, the transgene is selectively or exclusively expressed at a particular location within the transformed plant, e.g. within the plant leaves.

A “stably transformed” plant has generally been selected and regenerated following transformation. The changes caused by the transformation process in a stably transformed plant are passed to reproductive structures and progeny, and the phenotype of the parent stably transformed plant is thus expressed in offspring. Larger fragments or even entire genes can be used to produce stable transgenic plants. Alternatively, multiple paired gene fragments may be used together to produce stable transformants.

A “transformed plant” generally refers to a plant, a plant cell, plant tissue, seed, progeny thereof, or any other part of a plant that has been through, or is derived from a plant that has been through, a transformation process in which at least one foreign polynucleotide sequence is introduced into the plant. As used herein, a “plant” or “transformed plant” includes any and all portions and life stages of the plant e.g. seeds, grains, fruit, flowers, roots, tissue, cells (e.g. within and/or removed from the plant), stalks, leaves, etc, as well as progeny of the plant. Transgenic lines are typically established from seed of the transformed plant that are homozygous for the foreign polynucleotide in all tissues and transmit it to their progeny.

“Nucleic acid” and terms associated therewith (e.g. DNA, RNA, polynucleotide, oligonucleotide, etc.) has the meaning as is typically understood in the art, as do the terms protein, polypeptide, peptide, recombinant polynucleotide, recombinant polypeptide, etc. (e.g. see U.S. Pat. No. 8,633,353, the complete contents of which is herein incorporated by reference in entirety). A “synthetic” oligonucleotide or polypeptide sequence is one that is created by polymerization of isolated building blocks (nucleotides or amino acid residues) using chemical synthesis methods known in the art.

A “nucleic acid construct” is a nucleic acid sequence (DNA, RNA) comprising (or capable of comprising) a “foreign” (non-native, exogenous, heterologous, etc.) nucleic acid sequence of interest. The sequence of interest may encode an RNA sequence and/or a polypeptide of interest. The encoding sequence may be under transcriptional control of one or more transcriptional elements, e.g. a promoter that is operably linked to the coding sequence, various enhancer sequences, etc. and the construct may encode other molecules of interest such as detectable tagging sequences, targeting or signal sequences that direct a gene product to a particular location, etc. For the present invention, the promoter is generally capable of driving expression of a nucleic acid sequence of interest within a plant, e.g. within a true grass, and may be capable of directing expression of the nucleic acid sequence of interest within a particular location within the plant, e.g. within leaf cells. In some aspects, the sequence(s) of interest include(s) one or more rust fungal sequences which are associated with pathogenicity of a fungus.

“Gene” or “gene sequence” refers to the partial or complete coding sequence of a gene, and/or its complement, and/or its 5′ or 3′ untranslated regions. A gene is also a functional unit of inheritance, and in physical terms is a particular segment or sequence of nucleotides along a molecule of DNA or RNA which may or may not be involved in producing a polypeptide chain.

An “isolated” macromolecule (e.g. polynucleotide or polypeptide) is more enriched in (or out of) a cell than in its natural state. Alternatively, or in addition, the isolated macromolecule may be purified, i.e. separated from other cellular components with which it is typically associated, e.g., by any of the various purification methods known in the art.

“Alignment” between macromolecules refers to a number of nucleotide bases or amino acid residue sequences aligned by lengthwise comparison so that components in common (i.e., nucleotide bases or amino acid residues at corresponding positions) may be visually and readily identified, e.g. to identify conserved sequences. The fraction or percentage of components in common is related to the homology or identity between the sequences, with identity referring to nucleotides/amino acids that are identical, and similarity referring to amino acids that are recognized as conservative substitutions with similar properties or characteristics, e.g. both are negatively charged, positively charged, aliphatic, etc.

A “conserved domain” or “conserved region” as used herein refers to a region in heterologous polynucleotide or polypeptide sequences where there is a relatively high degree of sequence identity or similarity between the distinct sequences. For nucleic acid sequences, a conserved domain is generally at least about nine base pairs (bp) in length and for a polypeptide sequence, a conserved domain is generally at least about 3 amino acids in length, but may be e.g. 5-10 or more amino acids in length. Conserved domains may be identified as regions or domains of identity in comparison to e.g. a consensus sequence by using alignment methods well known in the art.

The terms “paralog” and “ortholog” refer to evolutionarily related genes that have similar sequences and functions. Orthologs are structurally related genes in different species that are derived by a speciation event. Paralogs are structurally related genes within a single species that are derived by a duplication event. The term “equivalog” describes members of a set of homologous proteins that are conserved with respect to function since their last common ancestor. Related proteins are grouped into equivalog families, and otherwise into protein families with other hierarchically defined homology types. This definition is provided at The Institute for Genomic Research (TIGR) World Wide Web (www) website, “tigr.org” under the heading “Terms associated with TIGRFAMs”.

In general, the term “variant” refers to molecules with some differences, generated synthetically or naturally, in their base or amino acid sequences as compared to a reference (native) polynucleotide or polypeptide, respectively. These differences include substitutions, insertions, deletions or any desired combinations of such changes in a native polynucleotide of amino acid sequence. “Allelic variant” or “polynucleotide allelic variant” refers to any of two or more alternative forms of a gene occupying the same chromosomal locus. “Allelic variant” and “polypeptide allelic variant” may also be used with respect to polypeptides.

In general, the term “derivative” refers to the chemical modification of a nucleic acid molecule or amino acid sequence. Chemical modifications can include replacement of hydrogen by an alkyl, acyl, or amino group or glycosylation, pegylation, or any similar process that retains or enhances biological activity or lifespan of the molecule or sequence.

The invention thus provides transformed or genetically engineered plants that are resistant to infection by rust fungi. The plants are generally members of the monocot family Poaceae (also called Gramineae or true grasses), a large and ubiquitous family of monocotyledonous flowering plants that are cultivated for many purposes. For example, this family includes cereal crops which are typically cultivated for the edible components of their grain (caryopsis), although other portions or components of the plants are also of high value e.g. as animal feed, for ethanol production, etc.

Exemplary true grasses that may be rendered rust-resistant by the procedures described herein include but are not limited to: maize, various types of wheat, barley, sorghum, millet, oats, triticale, rye, teff, sugarcane, and annual or perennial grasses used for biomass, forage or turf.

Methods and protocols for transiently silencing one or more genes of interest in a plant are known in the art, and include, but are not limited to: viral transduction of plants, e.g. using an RNA virus such as Barley Stripe Mosaic Virus to carry out BSMV-Virus Induced Gene Silencing (BSMV VIGS), Agrobacterium transformation of localized tissues and electroporation or biolistic transformation of individual cells.

Methods and protocols to stably silence one or more genes of interest in a plant are known in the art, and generally include expression of antisense or double stranded RNAs molecules homologous to transcripts of the genes to be silenced. Delivery of the constructs expressing the RNAs to make stable transgenic lines include, but are not limited to: protoplast transformation via polyethylene glycol (PEG) fusion, electroporation, microinjection, etc. using a nucleic acids or a nucleic acid construct; microprojectile bombardment using DNA coated particles, or projectiles of bacteria, yeast phage, or agrobacterium; or various technologies where Agrobacterium tumefaciens is used to deliver the constructs into various cell types.

In the practice of the present invention, the one or more genes of interest that are silenced “in” a transgenic plant of the invention may be, but are generally not, plant derived genes. Rather, they are genes that would otherwise be expressed by a pathogen that invades the plant. Silencing of the genes prevents (or inhibits, slows, attenuates, decreases, lessens, etc.) the pathogenic process that usually occurs and would otherwise occur in the plant after infection or infestation by the pathogen. For example, symptoms that may be prevented or decreased include but are not limited to the amount of fungal spread within the plant after infection, the number or size of the uredinia or other spore producing structures, the rate at which uredinia develop, the number of fungal spores that are produced or the frequency that spores can infect the plants. Silencing of the pathogen genes thus prevents the development of a full-blown infection of the plant by the pathogen. Those of skill in the art will recognize that in some instances, an infective process may be completely thwarted by gene silencing, i.e. no symptoms of infection will be detectable in the plant. However, benefit may also accrue if the infection is only partially decreased or slowed, compared to a control plant in which the genes of interest are not silenced by the methods described herein. In polycyclic diseases like the cereal rust diseases, reductions in spore infection, establishment or spread in the plant and reductions in the amount of spores produced, all decrease the rate at which epidemics occur in the field.

In some aspects, the genes that are silenced are pathogen genes that are associated with pathogenicity of the pathogen, i.e. that are associated with the ability of the pathogen to e.g. reproduce, to express proteins/polypeptides that are necessary for reproduction and/or for spreading from cell to cell within the plant, and/or for passage from one plant to another, etc.

The host plants that may be transformed as described herein are described above. In some aspects, the genes that are silenced within the host plants are silenced in all cells of the plant or in specific tissues like leaves or stems.

The genes that are silenced by the methods described herein are generally genes of a pathogenic organism. Exemplary pathogenic organisms include but are not limited to: fungi such as rusts, (e.g. Puccinia species).

In some aspects, the genes that are silenced are fungal genes and are Puccinia genes from one or more of P. graminis f. sp. tritici (Pgt), P. triticina (Pt), and P. striiformis f. sp. tritici (Pst). Exemplary genes include but are not limited to P. graminis f. sp. tritici (Pgt) genes PGTG_{—}11658, PGTG_{—}01136, PGTG_{—}03590, PGTG_{—}01215, PGTG_{—}03478, PGTG_{—}01304, PGTG_{—}07754, PGTG_{—}12890, PGTG_{—}14350and PGTG_{—}16914, as well as variants, derivatives, orthologs and paralogs thereof. In particular, the genes PGTG_{—}11658, PGTG_{—}01136 and PGTG_{—}01215, which are able to confer resistance to multiple Puccinia species that infect different grasses, are used.

The invention also encompasses products produced by or made or derived from the transformed plants or portions of the transformed plants of the invention, e.g. grains, oils, flours, extracted or processed carbohydrates (such as molasses, bagasse, sugar, etc. from sugarcane, forage and hay crops, crops produced for biomass, such as those used for bioenergy production, ornamental and turf grasses and grasses used for environmental uses such as erosion control.

### EXAMPLES

### Example 1

### Materials and Methods

### Plant Materials, Growth Conditions, and Fungal Races

Wheat cultivars McNair 701, Zak, and Fielder were used in these studies. McNair 701 was used for Barley Stripe Mosaic Virus-Virus Induced Gene Silencing (BSMV VIGS) experiments for stem rust and leaf rust, Zak was used for stripe rust. Seedlings for Virus Induced Gene Silencing (VIGS) assays were sown in pots containing potting mix and placed in growth chambers with temperatures of 24° C. during the day and 20° C. at night, 23 to 50% relative humidity, and 16 h of light. Plants were watered daily and fertilized with a dilute nutrient solution weekly. Urediniospores from P. graminis pv. tritici (race Pgt7A) and mixtures of races of P. triticina were increased on McNair 701, and urediniospores from P. striiformis (race Pst78) were increased on Zak. Fresh spores were collected and used in inoculation experiments.

Isolation of RNA from Haustoria and Infected Wheat Leaves of P. graminis

Haustoria were isolated from heavily stem rust infected wheat leaves at 4-5 dpi (just prior to sporulation) using ConA affinity chromatography (Yin et al. 2009). 4-5 dpi stem rust infected leaves and haustorial cells were ground separately in a mortar in liquid nitrogen. Total RNA was isolated from frozen powder using the Qiagen Plant RNeasy® kit (Qiagen, Chatsworth, Ga.) according to the manufacturer's instruction. The quantity and purity of isolated total RNA was analyzed by 2% agarose gel electrophoresis as well as by using a spectrophotometer.

### Sequence Analysis and Conserved Gene Screening

Approximately 20 million sequence reads were obtained from RNAs of haustorial and infected leaf libraries. The number of sequence reads for each gene in the haustorial and whole-leaf RNAseq libraries was compared. Only genes that were at least two times more frequent in the haustorial sequences than the whole leaf sequences were used. This provided a gene set of 1036 genes from P. graminis pv. tritici. Genes from this collection were then selected on the basis of sequence conservation among the rust fungi. 583 genes had clear homologs in all three wheat-infecting rust species as determined by standard homology searches (E score cut off 0.00). This conserved haustorial gene set was used for functional analysis.

Construction of BSMV-Derived Vectors and in vitro Transcription of Viral RNAs

Barley Stripe Mosaic Virus (BSMV)-derived constructs were made by methods described previously (Yin et al. 2011). Briefly, selected fragments of the targeted 583 genes were amplified from infected leaf cDNA of stem rust Pgt7A or stripe rust Pst78 using primers listed in Table 3. The amplicons were directionally ligated into NotI/PacI sites of the BSMV gamma vector. All constructs were verified by DNA sequencing. Each resulting viral genome included one antisense strand of one of 88 of the 583 fungal transcripts. The negative control Barley Stripe Mosaic Virus-Multiple Cloning Site (BSMV::MCS) carried only a 121-bp antisense fragment of the MCS from pBluescript K/S and carried no Puccinia spp. sequences (Yin et al. 2011). In vitro transcripts of viral RNAs were prepared from three linearized plasmids using the mMessage mMachine® T7 in vitro transcription kit (Ambion, Austin, Tex., U.S.A.) following the manufacturer's instructions.

### Virus Inoculations, Rust Inoculations, and Disease Assays

Each of the experimental plants was infected with one of the 90 BSMV-derived constructs or the control construct as described previously (Yin et al. 2011). Briefly, mixtures of equal amounts (2.5 μl) of each of the three BSMV viral RNAs required for viral infection of plant cells and 45 μl of FES buffer (77 mM glycine, 60 mM K₂HPO₄, 22 mM Na4P₂O₇.10H₂O, 1% [wt/vol] bentonite, and 1% [wt/vol] celite) were applied to wheat seedlings (ten-days-old) by rub inoculation. Seedlings were then lightly misted with water and returned to the growth chamber. After approximately 10 days, inoculated plants developed symptoms of BSM virus infection, demonstrating that the BSMV constructs were being expressed in the plants. The third and fourth leaves of each plant were then inoculated with fresh spores of stem rust isolate race Pgt7A or Pst78 or Pt in order to test whether or not the rust fungal genes being expressed by the BMV would have an impact on the ability of the stem rust to infect the plants. A decrease in infection would be consistent with silencing of a rust gene via hybridization of mRNA transcribed from the rust gene with an RNA produced from the BSM virus, thereby preventing successful translation of the rust gene product (e.g. a protein or polypeptide). If no effect on infection was observed, either gene silencing did not take place, or gene silencing did take place but the gene that was silenced was probably not essential to infection.

Twelve days after inoculation with rust, the infection types were assessed based on a 0-4 rating scale for stem rust and leaf rust (Stakman et al. 1962). Twenty days after inoculation with stripe rust, the infection types were assessed based on a 0-9 scale (Line and Qayoum 1992). Any VIGS constructs that reduced the speed or amount of rust reproduction were reexamined several times to check consistency.

### Statistical Analysis

Pathogenicity analysis after gene silencing was performed using JMP Version 4.0 (SAS Inc, Cary, N.C.).

### Results

### Transient Suppression Assays of Conserved Haustoria-Specific Genes From Stem Rust Fungi by VIGS Assays

Eighty-eight rust genes were selected to make VIGS constructs and used to inoculate wheat. Ten days after inoculation with the various VIGS constructs the wheat was infected with stem rust fungus isolate 7a and the amount of rust development and sporulation were assessed after 12 days.

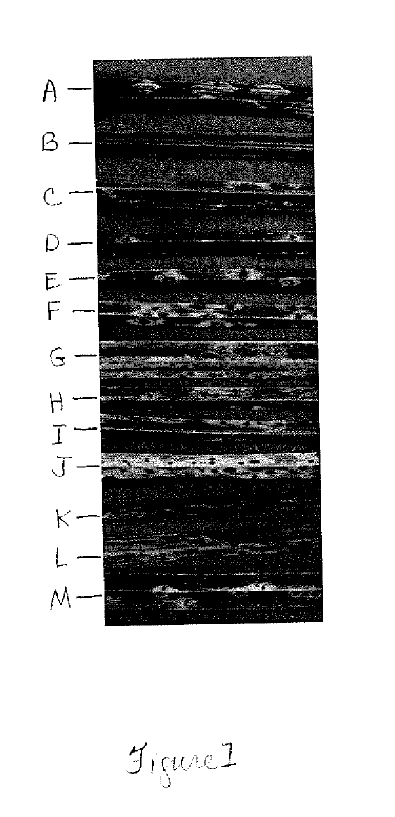

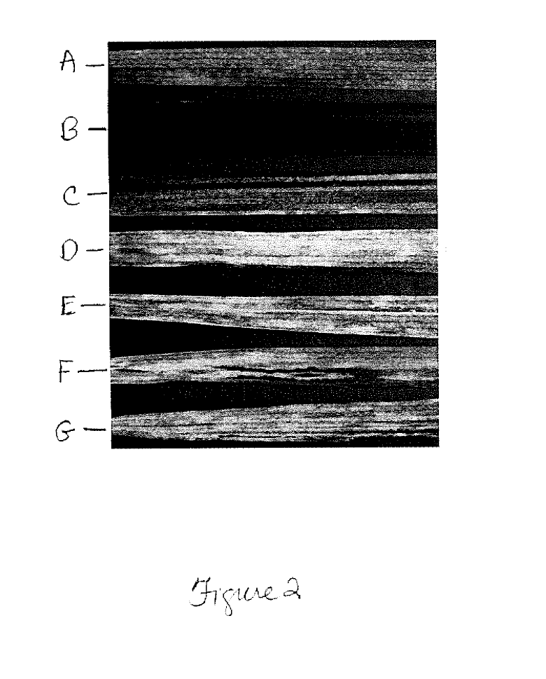

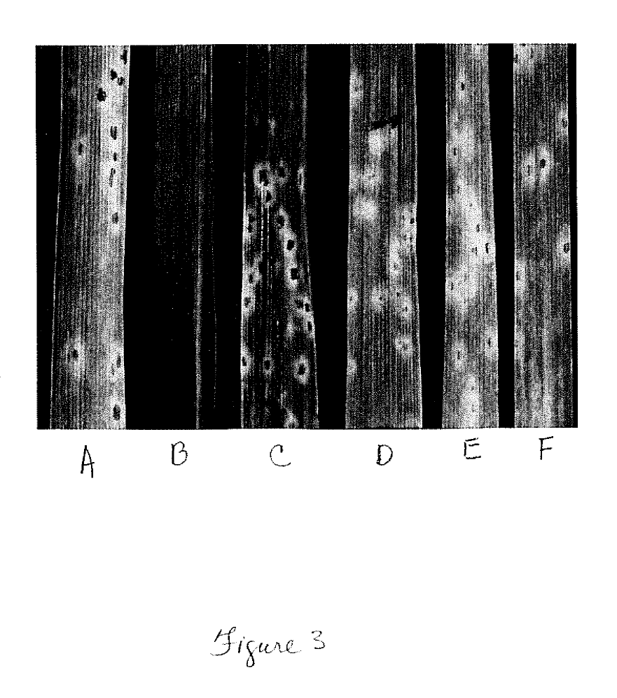

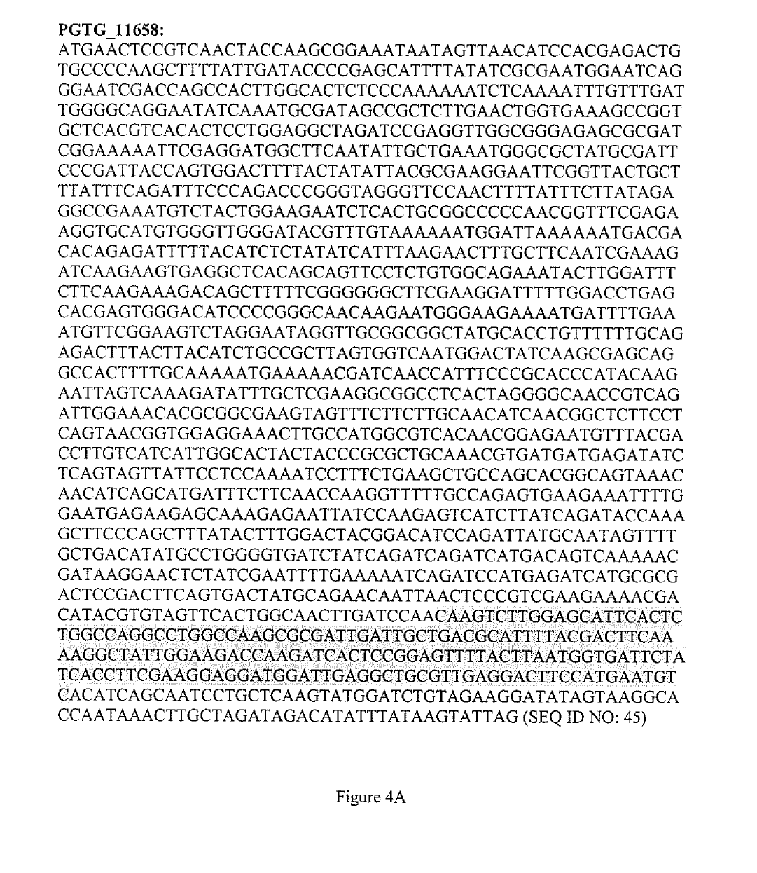

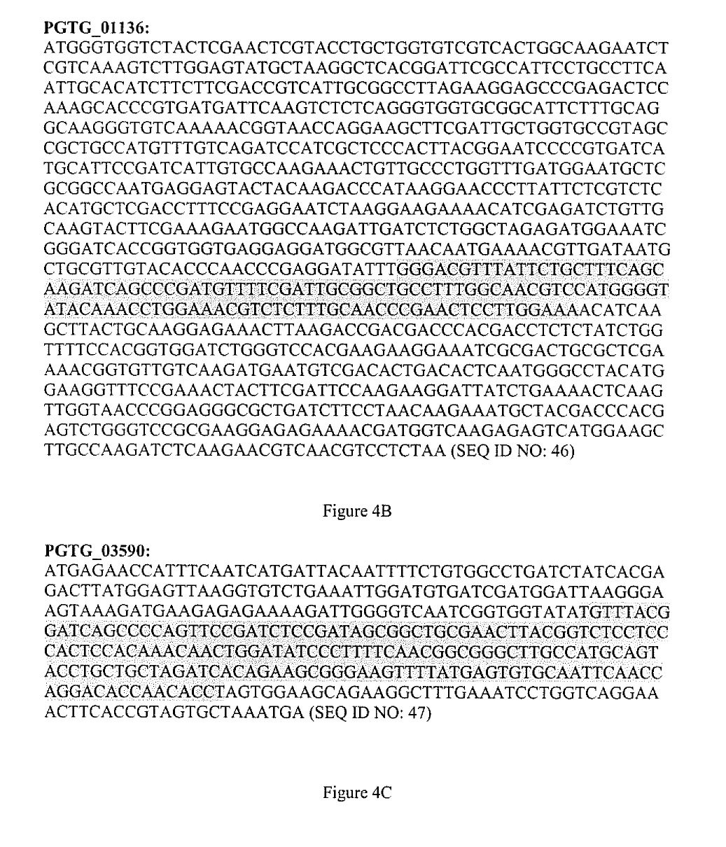

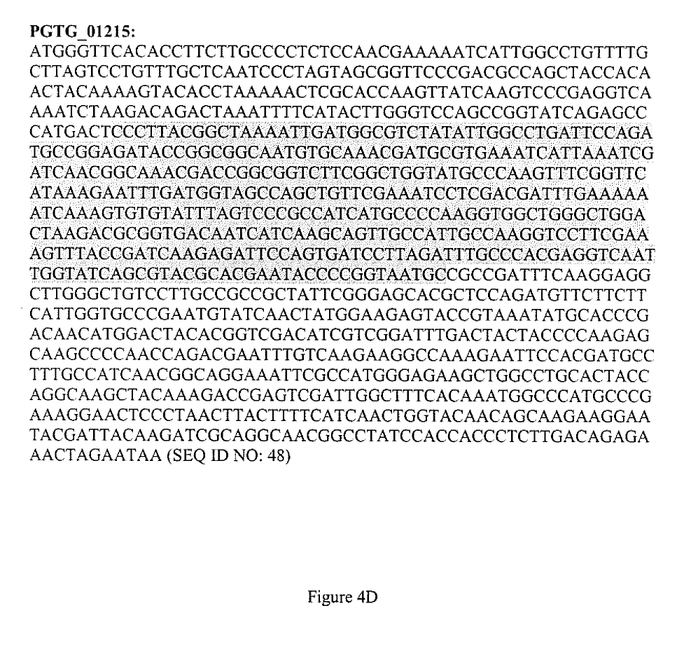

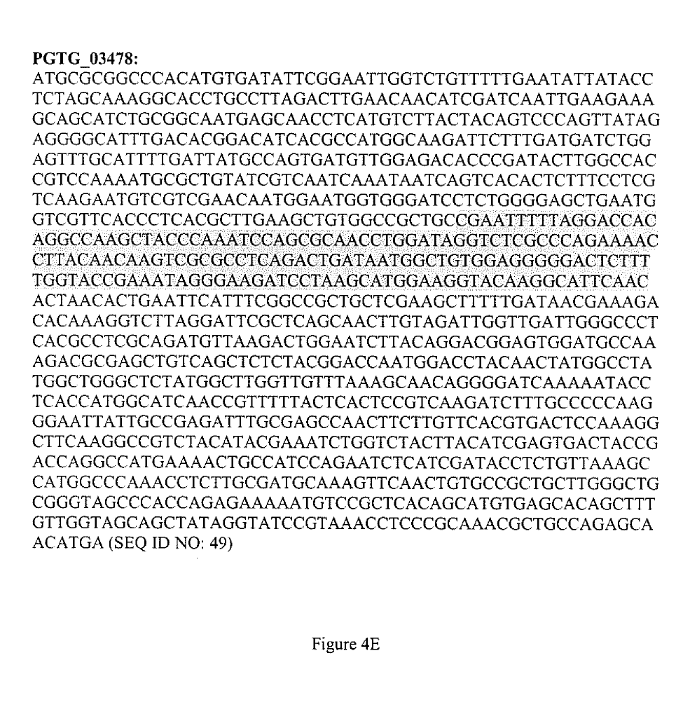

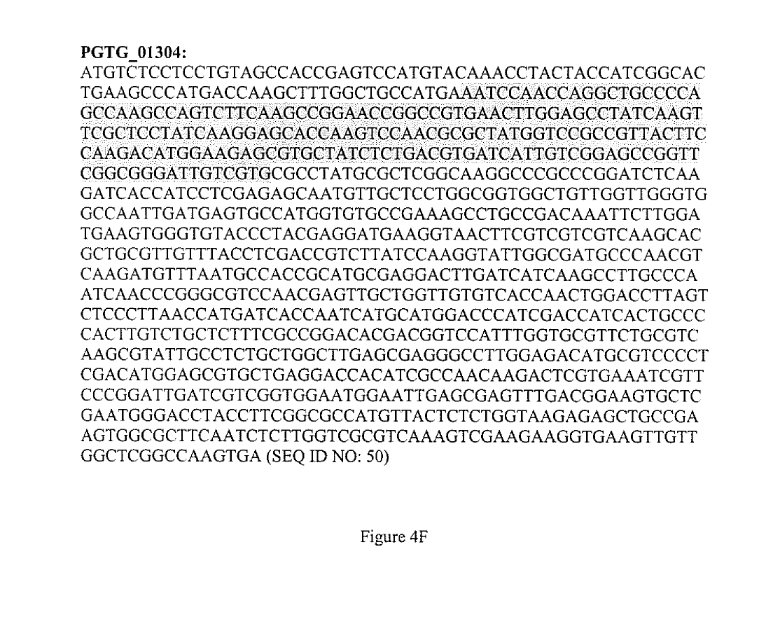

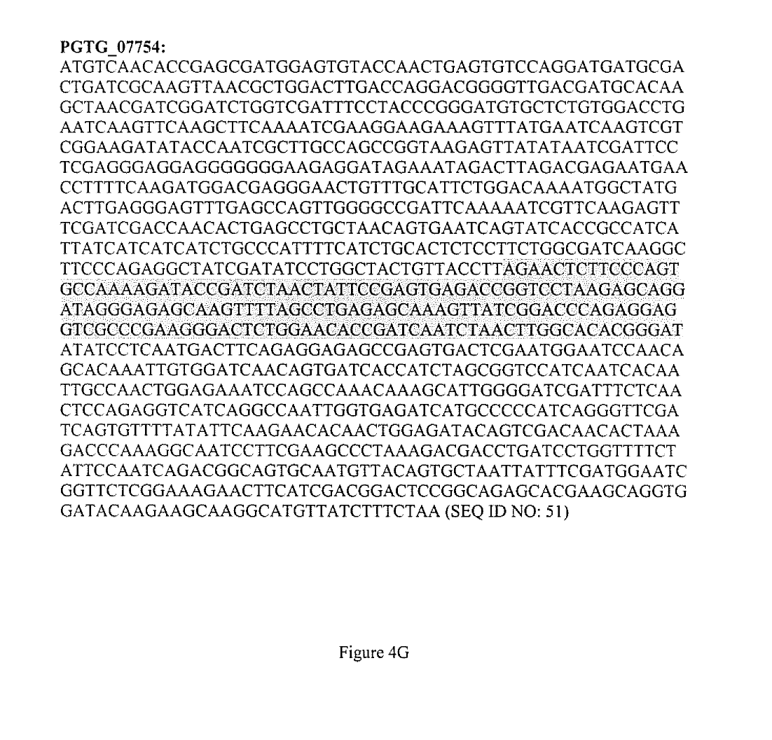

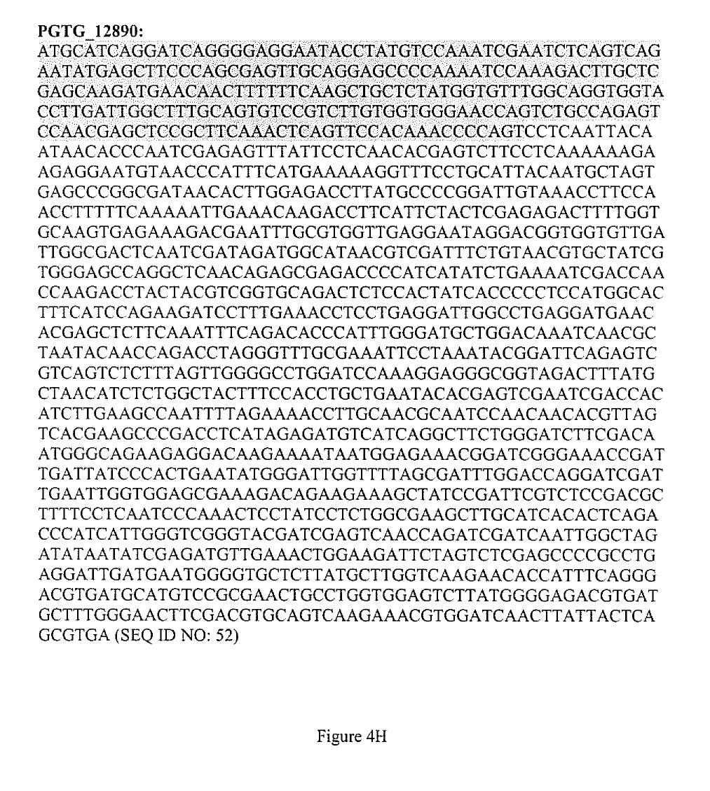

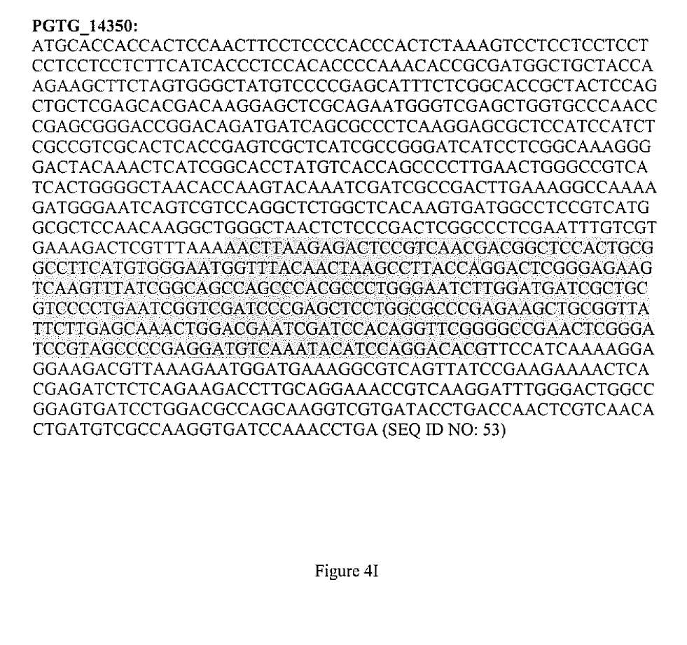

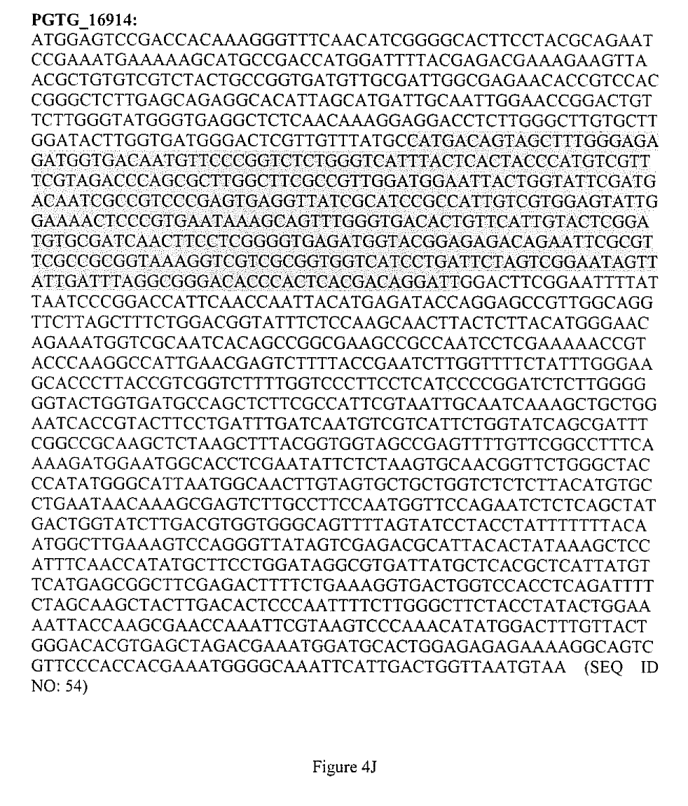

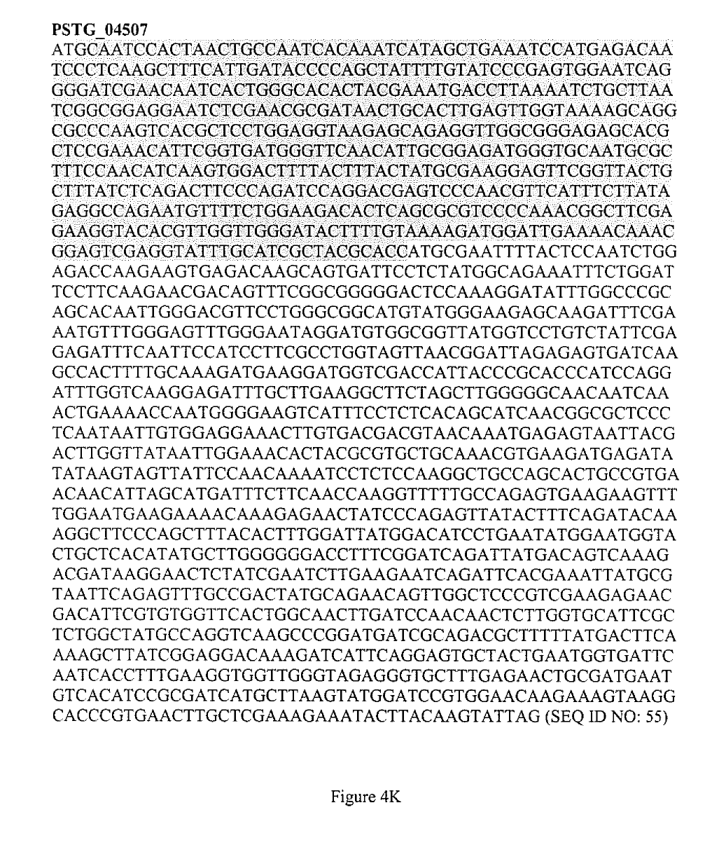

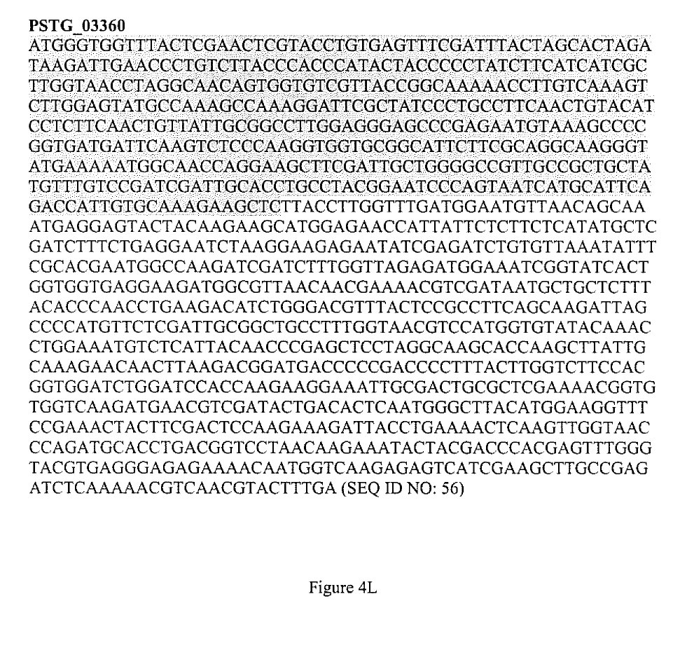

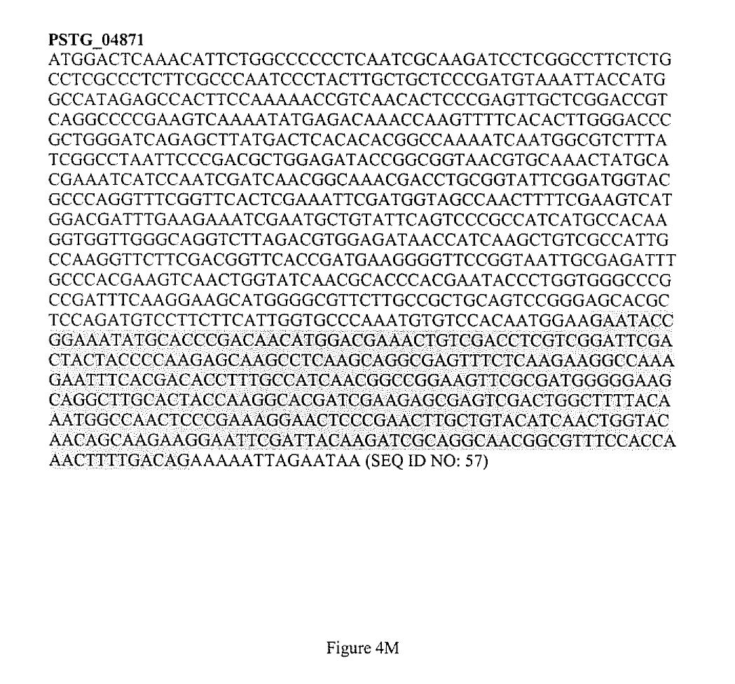

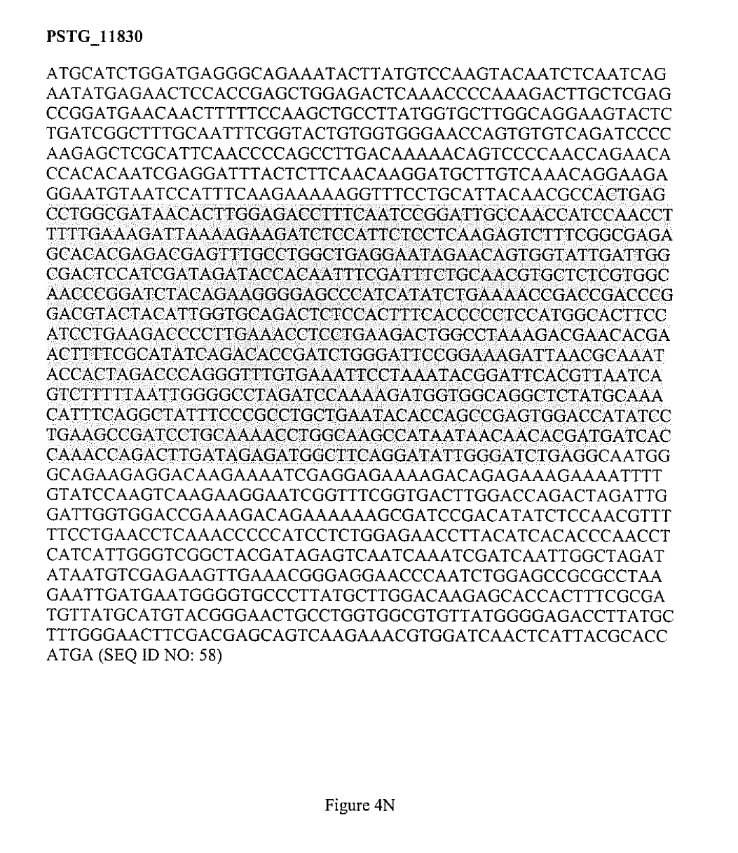

Ten genes constructs reduced the pathogenicity of the rust fungus by various amounts (FIG. 1, Table 2). The other 78 genes showed no noticeable effects on rust pathogenicity or reproduction. Table I shows the expression ratios of the 10 genes in purified haustoria (Haust) vs. infected wheat leaves (InfW) in an RNAseq experiment. The same VIGS constructs of eight of the ten genes were inoculated on wheat cultivar Zak, then infected with stripe rust isolate Pst78 and the amount of rust development and sporulation were assessed after 20 days. Four gene constructs reduced the pathogenicity of the rust fungus at different levels (Data not shown). The four genes were selected to make stripe rust specific VIGS constructs to confirm the results. The results were consistent with the stem rust fungus gene constructs (FIG. 2, Table 2). These four stripe rust specific VIGS constructs were used to infect wheat cultivar 701, which was then infected with the leaf rust fungus and the amount of rust development and sporulation was assessed after 12 days. Three of the stripe rust gene constructs suppressed the pathogenicity of leaf rust (FIG. 3, Table 2).

TABLE 1
P. graminis pv. tritici genes that reduce pathogenicity when they are
silenced in the pathogen by VIGS expression in the wheat plant.

| | Predicted function | |
|---|---|---|
| | based on homology to | Expression |
| Gene ID^{a} | database sequences | (Haust/InfW^{b}) |
| PGTG_11658 | tryptophan | 7.7 |
| | 2-monooxygenase: IaaM | |
| PGTG_01136 | fructose-bisphosphate | 5.84 |
| | aldolase | |
| PGTG_03590 | predicted secreted protein | 81.78 |
| PGTG_01215 | family 26 glycoside | 5.8 |
| | hydrolase (predicted | |
| | secreted protein) | |
| PGTG_03478 | family 76 glycoside | 7.26 |
| | hydrolase (predicted | |
| | secreted protein) | |
| PGTG_01304 | pathogen-induced | 2.11 |
| | defense-responsive | |
| | protein 8 | |
| PGTG_07754 | glucosyl transferase | 6.63 |
| PGTG_12890 | predicted protein, possible | 7.12 |
| | Proteophosphoglycan | |
| PGTG_14350 | possible ABC | 2.41 |
| | transporter-like (predicted | |
| | secreted protein) | |
| PGTG_16914 | amino-acid permease inda1 | 15.41 |

^{a}gene prediction designation in Broad Institute database
^{b}ratio of expression in haustoria (Haust) vs. expression in infected leaf (InfW)

TABLE 2
Pathogenicity analysis after P. graminis pv. tritici genes
were silenced in the wheat by VIGS.

| | Silencing effects on Pathogenicity* | | |
|---|---|---|---|
| Gene ID | Pgt | Pst | Pt |
| PGTG_11658 | + | + | + |
| PGTG_01136 | + | + | + |
| PGTG_03590 | + | nt | nt |
| PGTG_01215 | + | + | + |
| PGTG_03478 | + | − | − |
| PGTG_01304 | + | − | nt |
| PGTG_07754 | + | − | − |
| PGTG_12890 | + | + | − |
| PGTG_14350 | + | nt | nt |
| PGTG_16914 | + | − | − |

Each gene silencing treatment was done three times
“+”: Reduce sporulation (p <0.001);
“−”: No significant reduction of sporulation;
“nt”: not tested.

TABLE 3
Primers used in construction of Barley
stripe mosaic virus-derived vectors and
vectors for stable transformation.

| Primers | Sequence (5′ to 3′) |
|---|---|
| PGTG_11658F | ATAAGAATGCGGCCGCTAAACTATCAAGTCTT |
| | GGAGCATTCACTCTGG (SEQ ID NO: 1) |
| PGTG_11658R | CCTTAATTAAGGGACATTCATGGAAGTCCTCA |
| | ACGC (SEQ ID NO: 2) |
| PGTG_01136F | ATAAGAATGCGGCCGCTAAACTATGGGACGTT |
| | TATTCTGCTTTCAG (SEQ ID NO: 3) |
| PGTG_01136R | CCTTAATTAAGGTTTCCAAGGAGTTCGGGTTG |
| | C (SEQ ID NO: 4) |
| PGTG_03590F | ATAAGAATGCGGCCGCTAAACTATTGTTTACG |
| | GATCAGCCCCAGTT (SEQ ID NO: 5) |
| PGTG_0359OR | CCTTAATTAAGGAGGTGTTGGTGTCCTGGTTG |
| | AA (SEQ ID NO: 6) |
| PGTG_01215F | ATAAGAATGCGGCCGCTAAACTATCCCTTACG |
| | GCTAAAATTGATGG (SEQ ID NO: 7) |
| PGTG_01215R | CCTTAATTAAGGGCATTACCGGGGTATTCGTG |
| | (SEQ ID NO: 8) |
| PGTG_03478F | ATAAGAATGCGGCCGCTAAACTATCGAATTTT |
| | TAGGACCACAGGCC (SEQ ID NO: 9) |
| PGTG_03478R | CCTTAATTAAGGGTTGAATGCCTTGTACCTTC |
| | CA (SEQ ID NO: 10) |
| PGTG_01304F | ATAAGAATGCGGCCGCTAAACTATAATCCAAC |
| | CAGGCTGCCCCA (SEQ ID NO: 11) |
| PGTG_01304R | CCTTAATTAAGGCACGACAATCCCGCCGAACC |
| | (SEQ ID NO: 12) |
| PGTG_07754F | ATAAGAATGCGGCCGCTAAACTATAGAACTCT |
| | TCCCAGTGCCAA (SEQ ID NO: 13) |
| PGTG_07754R | CCTTAATTAAGGATCCCGTGTGCCAAGTTAGA |
| | (SEQ ID NO: 14) |
| PGTG_12890F | ATAAGAATGCGGCCGCTAAACTATATGCATCA |
| | GGATCAGGGGAG (SEQ ID NO: 15) |
| PGTG_12890R | CCTTAATTAAGGACTGGGGTTTGTGGAACTGA |
| | (SEQ ID NO: 16) |
| PGTG_14350F | ATAAGAATGCGGCCGCTAAACTATAACTTAAG |
| | AGACTCCGTCAACG (SEQ ID NO: 17) |
| PGTG_14350R | CCTTAATTAAGGCGTGTCCTGGATGTATTTGA |
| | CA (SEQ ID NO: 18) |
| PGTG_16914F | ATAAGAATGCGGCCGCTAAACTATCATGACAG |
| | TAGCTTTGGGAGAG (SEQ ID NO: 19) |
| PGTG_16914R | CCTTAATTAAGGAATCCTGTCGTGAGTGGGTG |
| | T (SEQ ID NO: 20) |
| PSTG_04507F | CCTTAATTAAGGGCAATCCACTAACTGCCAAT |
| | CAC (SEQ ID NO: 21) |
| PSTG_04507R | ATAAGAATGCGGCCGCTAAACTATCATGGTGC |
| | GTAGCGATGCAAATA (SEQ ID NO: 22) |
| PSTG_03360F | CCTTAATTAAGGATGGGTGGTTTACTCGAACT |
| | CG (SEQ ID NO: 23) |
| PSTG_03360R | ATAAGAATGCGGCCGCTAAACTATGAGCTTCT |
| | TTGCACAATGGTCTG (SEQ ID NO: 24) |
| PSTG_04871F | CCTTAATTAAGGGAATACCGGAAATATGCACC |
| | CGAC (SEQ ID NO: 25) |
| PSTG_04871R | ATAAGAATGCGGCCGCTAAACTATCTGTCAAA |
| | AGTTTGGTGGAAACGC (SEQ ID NO: 26) |
| PSTG_11830F | CCTTAATTAAGGCACTGAGCCTGGCGATAACA |
| | CTT (SEQ ID NO: 27) |
| PSTG_11830R | ATAAGAATGCGGCCGCTAAACTATCCTCAGAT |
| | CCCAATATCCTGAAGC (SEQ ID NO: 28) |
| PGTG_11658STF | CACCAGCAACTTTTGCAAACATAAAAATGGTC |
| | GACCGGTCCCCGCACCCATACAAGACCTGGTC |
| | AAGGCAATCTGCTCGAAAGCCGCTTCACTAGG |
| | GGCAACCGTCAGAT (SEQ ID NO: 29) |
| PGTG_11658STR | ATAATCCAAAGTGTAAAGCTGGAAAGCCTTTG |
| | TATCTGAAAGTATAACTCTGGGATAGTTCTCT |
| | TTGACCTCTTCATTCCAAAACTTCTTCACTCT |
| | GGCAAAAACCTTGG (SEQ ID NO: 30) |
| PGTG_01136STF | CACCGCATTTGGCAACGTCCATGGCGTGTACA |
| | AGCCTGGGAATGTCTCCTTGCAGCCCGAACTT |
| | CTTGGCAAGCACCAAGCTTACTGCATTCTTTG |
| | CAGGCAAGGGTGTC (SEQ ID NO: 31) |
| PGTG_01136STR | TTGAGTGTCAGTATCGACGTTCATCTTGACCA |
| | CACCGTTTTCGAGCGCAGTCGCAATTTCCTTC |
| | TTGGTGGATCCAGATCCACCGTGGCGGAATGC |
| | ATGATCACGGGGAT (SEQ ID NO: 32) |
| PGTG_03590STF | CACCGAGAGAAAAGATTGGGGTCAATC |
| | (SEQ ID NO: 33) |
| PGTG_03590STR | TTTGTGGAGTGGGAGGAGACC |
| | (SEQ ID NO: 34) |

### Example 2

### Characterization of a Tryptophan 2-Monooxygenase Gene From Puccinia graminis f. sp. tritici Involved in Auxin Biosynthesis and Rust Pathogenicity: HIGS (Host-Induced Gene Silencing)

The plant hormone indole-3-acetic acid (IAA) is best known as a regulator of plant growth and development, but its production can also affect plant-microbe interactions. Microorganisms, including numerous plant-associated bacteria and several fungi, are also capable of producing IAA. The stem rust fungus, Puccinia graminis f. sp. tritici (Pgt), induced wheat plants to accumulate auxin in infected leaf tissue. A gene (Pgt-IaaM) encoding a putative tryptophan 2-monooxygenase, which makes the auxin precursor indole-3-acetamide (IAM), was identified in the Pgt genome and found to be expressed in haustoria cells in infected plant tissue. Transient silencing of the gene in infected wheat plants indicated it was required for full pathogenicity. Expression of Pgt-IaaM in Arabidopsis caused a typical auxin expression phenotype and promoted susceptibility to the bacterial pathogen Pseudomonas syringae pv. tomato DC3000.

Microorganisms are also capable of producing IAA. Tryptophan has been identified as a main precursor for IAA biosynthesis pathways in bacteria. The indole-3-acetamide (IAM) pathway is the best characterized, but not the only, pathway and includes two distinct steps. Tryptophan is first converted to IAM by the enzyme tryptophan-2-monooxygenase (IaaM) (encoded by IaaM gene) and then IAM is converted to IAA by an IAM hydrolase (IaaH) (encoded by the IaaH gene). The IAM pathway is mainly found in plant-associated bacteria, such as Agrobacterium tumefaciens, Pseudomonas syringae, P. savastanoi, and Pantoea agglomerans. A few fungi, such as Colletotrichum gloeosporioides and Fusarium proliferatum, also produce IAA via the IAM pathway. The IAA produced by bacterial pathogens is important for pathogenesis. Pathogenic strains of Erwinia herbicola were found to use the indole-3-acetamide (IAM) pathway for the production of IAA, whereas nonpathogenic strains were devoid of this pathway. Deactivating the IAM pathway by disrupting either the IaaM or Mall genes reduced the virulence of E. herbicola pv. gypsohhilae on Gypsophila paniculata. IAA has been also demonstrated to promote gall formation. An IAA deficient mutant of Pseudomonas savastanoi did not produce galls on host plants, but the ability to produce galls was restored when the mutant was transformed with genes for IAA synthesis. In contrast, multiple mutants of Ustilago maydis greatly reduced IAA levels but were still pathogenic and caused gall formation on maize similar to wild type strains. Other studies have demonstrated that IAA has functions in fungi that are independent of interactions with plants. For example, IAA reduced the ‘spore density effect’ on germination in Neurospora crassa. Exogenous IAA induced pseudohyphal growth in Saccharomyces cerevisiae and hyphal growth in Candida albicans.

In order to facilitate infection, plant pathogens deliver numerous effector proteins into the plant cells to promote their survival and growth in the host environment by altering host-cell structure and function. Proteins from biotrophic fungi and oomycetes that are secreted from haustorial cells are potential protein effectors and sometimes interact with resistance gene proteins. While such protein effectors from biotrophic fungi have received considerable attention, small molecule effectors, such as hormones, have not. Haustorial expression of biosynthetic enzymes for metabolites with biological activity in plant cells may be an indication of small molecule effectors in these fungi. The purpose of this study was to characterize a Puccinia graminis f. sp. tritici (Pgt) gene encoding a putative tryptophan 2-monooxygenase, which is highly expressed in rust haustoria cells and potentially involved in auxin biosynthesis and rust pathogenicity.

### Results

### A Puccinia graminis Gene Encodes a Putative Tryptophan 2-Monooxygenase

Auxins have been observed to increase in rust infected wheat tissues although the control of this increase is not known. A Pgt gene, PGTG_{—}11658 (herein Pgt-IaaM), encoding a predicted 588-amino acid tryptophan 2-monooxygenase was found in the Broad Institute Puccinia database (see the website located at www.broadinstitute.org). It had 82% amino acid identity with a predicted protein (PTTG_{—}06071) from P. triticina and 79% identity with a predicted protein (CQM-04507) in P. striiformis. In contrast, no homologous Melampsora sequences were identified. Homologous genes were identified in several non-rust fungi including several Fusarium species, Glomerella graminicola, Colletotchricum gloeosporioides and Neofusicoccum parvum. The genes in the three Puccinia species were found on sequence contigs carrying several common genes indicating they are on partially syntenic chromosome regions of at least 60-100 Kb. The partially syntenic region included genes encoding a mannose-1-phosphate guanyltransferase, a serine/threonine protein kinase and several hypothetical proteins. In bacteria and fungi where the IAM pathway has been characterized, the genes encoding the two catalytic enzymes are often adjacent to each other in the genomes. However, IaaM homologs were identified in the three Puccinia species, while IaaH homologs were not present on the chromosomes near these genes or anywhere else in the assembled genomes.

### Regulation of Pgt-IaaM Expression During Rust Infection

Pgt-IaaM mRNA expression during rust development in plants was examined by RT-qPCR analysis. Relatively low transcript levels were detected in urediniospores of P. graminis. Transcript levels in fungal cells growing in infected leaves were estimated to be almost 200 times higher than in urediniospores suggesting that Pgt-IaaM expression is induced in the biotrophic growth phase (FIG. 5A). When transcript levels in purified haustorial cells were compared to total fungal cells in infected leaves, they were estimated to be approximately seven times higher in the haustorial cell preparations. While infected leaves contain haustoria in addition to other cell types, the higher levels in the haustorial preparations indicate the transcripts are much more highly expressed in haustoria. The transcripts therefore appear to be haustoria specific or highly enriched in haustorial cells. No amino terminal sequences were identified using the Signal P 4.1 or iPSORT to indicate the protein was secreted from the haustorial cells.

### Hormone Levels in Urediniospores and P. graminis Infected Plants

Hormone levels were measured in different rust-infection stages and rust urediniospores. Rust urediniospores harvested from greenhouse grown plants contained 1.46±0.55 ng/g fresh weight (FW) free IAA. When auxin levels were measured in infected wheat plant leaves, very little IAA (0.01±0.01 ng/g FW) was detected at 2 days after stem rust infection. IAA increased to 3.28±0.09 ng/g FW at 4 days post infection (dpi), with higher levels (4.92±0.66 ng/g FW) at 6 dpi. Only trace amounts of IAA (0.01-0.02 ng/g FW) were detected in healthy leaves of control plants (FIG. 6A). The levels of IAM were analyzed and no significant difference was observed in healthy and rust-infected wheat leaves over the same time course (data not shown).

Levels of ABA and trans-Zeatin were also measured for comparison. Urediniospores carried extremely low levels (FIGS. 6B and 6C) of both hormones, possibly due to contamination from the host tissues. ABA levels increased in infected plants over a time course similar to that of IAA; no noticeable increase at 2 dpi but increasing through 4 and 6 dpi. Patterns of trans-Zeatin accumulation were very different, with similar levels to the uninoculated control plants at 2 and 4 dpi but reduced levels at 6 dpi. Overall, the results show that by 6 dpi, when the rust fungus is well-spread through the host tissue, the leaves contain elevated levels of IAA and ABA compared to healthy plants and reduced levels of trans-Zeatin.

### Silencing of Pgt-IaaM by Host-Induced Gene Silencing (HIGS) Reduces the Pathogenicity of P. graminis

To investigate the function of Pgt-IaaM in the wheat stem rust interaction, silencing was conducted using Barley stripe mosaic virus (BSMV)-mediated HIGS. First and second leaves of 12-day-old wheat cultivar McNair 701 were rub inoculated with the transcripts from either the BSMV construct that carried an RNAi target region of Pgt-IaaM or the control virus consisting of the same vector without the fungal DNA fragment incorporated into the multiple cloning site of they genome. After 10 days, those leaves displaying mild virus symptoms (pale yellow stripes on the leaves) were inoculated with P. graminis isolate Pgt7A. Samples of the infected leaves were harvested at 5 days after rust inoculation (dpi) for RT-qPCR analysis to determine the extent of gene Pgt-IaaM silencing. The remaining infected plants were kept in the growth chamber and the infection types (IT) were scored at 12 dpi. The fungal disease phenotype displayed a reduction in the size of uredinia compared to BSMV: MCS controls. Using the 0-to-4 scale described by Stakman et al. (1962), the plants inoculated with the BSMV:Pgt-IaaM construct often exhibited moderately resistant (MR) phenotypes with rust infection types (IT) of 2 to 2+, but the control plants inoculated with BSMV:MCS or no BSMV consistently exhibited susceptible ITs of 4. Although some plants inoculated with the BSMV:Pgt-IaaM construct showed fully susceptible interactions, others showed the moderately resistant phenotype in every experiment while the control plants never did, and the average reaction type was significantly lower (P<0.05) in the BSMV:Pgt-IaaM infected plants than the control plants. The effect of the siRNA molecules on the Pgt-IaaM transcript levels was examined by RT-qPCR assays. A significant reduction in Pgt-IaaM transcript abundance was detected in infected wheat leaves at 5 dpi and the average levels of Pgt-IaaM expression in silenced plants were approximately 31% of the control plants (FIG. 5B). These results indicate Pgt-IaaM is required for full pathogenicity of P. graminis.

### Expression of Pgt-IaaM in Arabidopsis Displays Pleiotropic Auxin-Related Phenotypes

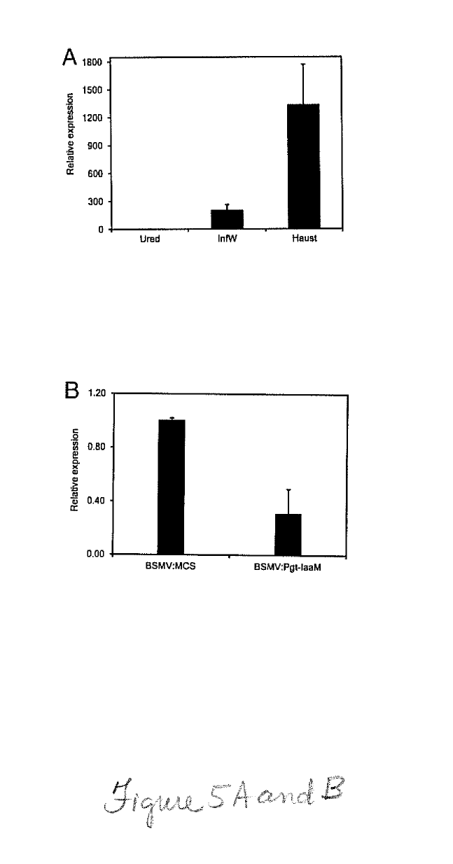

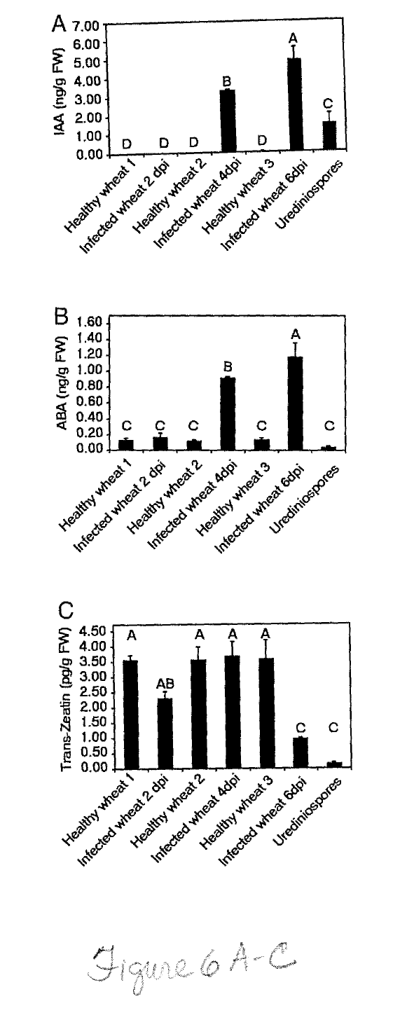

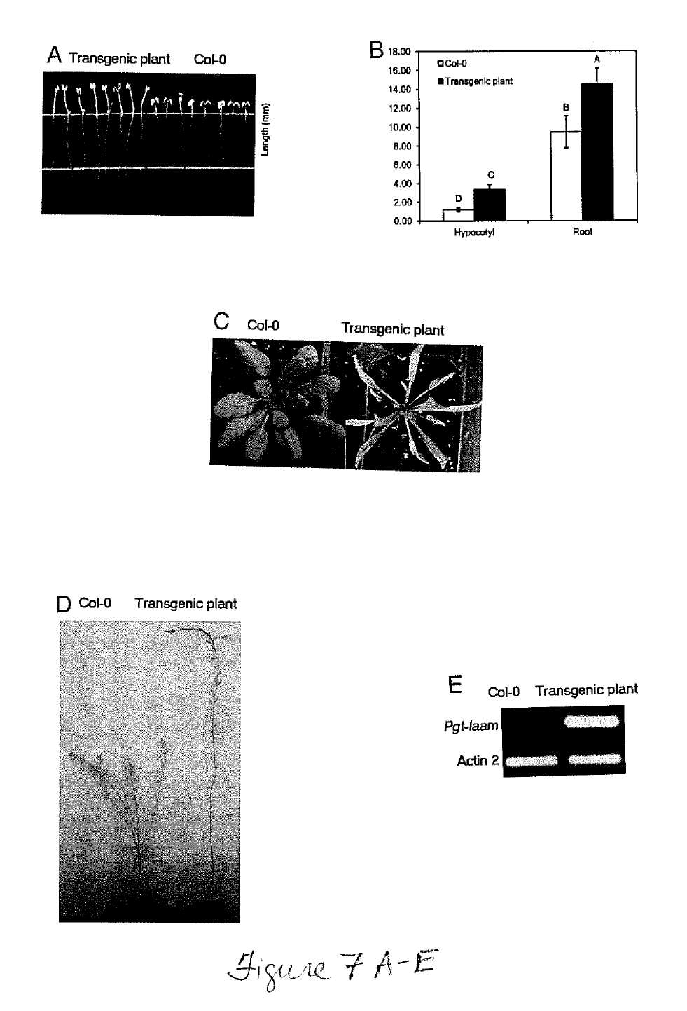

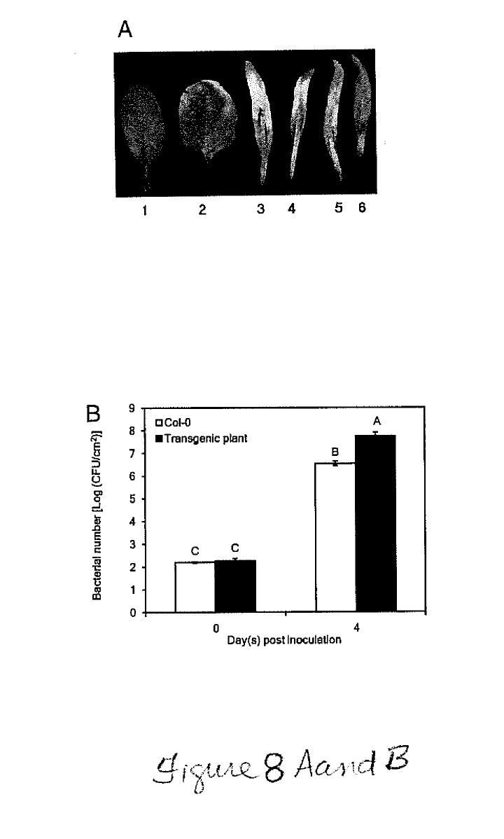

Pgt-IaaM was predicted to code for tryptophan 2-monooxygenase, an enzyme that catalyzes the first step in the IAM pathway to synthesize auxin from tryptophan. To determine if it is functional in auxin biosynthesis and whether expression of a single protein would increase auxin production in a plant, the gene was expressed in Arabidopsis thaliana biotype Columbia-0 (Col-0). The full predicted coding region was amplified from cDNA and inserted into a binary vector pCHF3 that controls transcription with a CaMV 35S promoter. Three independent transgenic Arabidopsis lines that expressed the gene were generated by Agrobacterium-mediated transformation. All three 35S: Pgt-IaaM transgenic lines displayed pleiotropic auxin-related phenotypes (FIG. 7). The 5-day-old transgenic seedlings exhibited approximately 3 fold longer hypocotyls and 1.5 fold longer primary roots than wild type Col-0 (FIG. 7A and 7B). Four-week-old transgenic plants displayed narrow and downward-curling leaves (FIG. 7C). The transgenic plants also exhibited strong apical dominance and the height of fully grown transgenic plants was approximately twice that of wild type plants (FIG. 7D). In addition, adult transgenic plants had twisted inflorescence stems and reduced seed set in many siliques (data not shown). RT-PCR analysis confirmed that phenotypes observed in 35S: Pgt-IaaM transgenic lines resulted from the accumulation of Pgt-IaaM transcript (FIG. 8E). IAA and IAM levels were measured in wild type plants and 35S: Pgt-IaaM transgenic plants at different developmental stages and tissues. The free IAA levels in transgenic plants were higher than wild type plants in all the tissues tested. Ten-day-old seedlings, 4-week-old rosette leaves, 6-week-old cauline leaves and flowers of transgenic plants contained approximately six, eight, three and two times more free IAA than wild type, respectively (Table 4). Similar to that of IAA, IAM levels in transgenic plants were much higher than wild type plants in all the tissues tested. Four-week-old rosette leaves, 6-week-old cauline leaves and flowers of transgenic plants contained approximately 20, 14, three and 36 times more JAM than wild type, respectively. The IAM level of 10-day-old seedlings in non-transgenics was undetectable but trace amounts were detected in the other tissues tested (Table 4). These results indicate that the Pgt-IaaM gene functions in auxin synthesis.

TABLE 4
IAA and IAM levels in different developmental stages of wild type
Arabidopsis (Col-0) and transgenic plants.

| | | Free IAA | IAM |
|---|---|---|---|
| Plants | Organ tissues | (pg/g FW) | (ng/g FW) |
| Col-0 | 10-day-old seedlings | 2.45 ± 1.22 | 0.00 ± 0.00 |
| Transgenic plant | | 15.53 ± 5.79 | 2.37 ± 0.24 |
| Col-0 | 4-week-old rosette | 8.85 ± 7.11 | 0.17 ± 0.06 |
| Transgenic plant | leaves | 74.69 ± 11.85 | 3.36 ± 0.73 |
| Col-0 | 6-week-old cauline | 16.63 ± 5.62 | 0.42 ± 0.17 |
| Transgenic plant | leaves | 54.96 ± 16.94 | 5.71 ± 0.18 |
| Col-0 | Flowers | 130.28 ± 39.09 | 0.16 ± 0.08 |
| Transgenic plant | | 249.60 ± 47.30 | 5.72 ± 0.57 |

Specified organ tissues from Col-0 and transgenic plants were harvested and used for IAA and IAM measurement.
Values are means and SEs of three replicates of Col-0 and a single transgenic line.
Three independent transgenic lines were investigated with similar results.

### Expression of Pgt-IaaM in Arabidopsis Promotes Susceptibility to the Bacterial Pathogen Pseudomonas syringae pv. tomato DC3000 (Pst DC3000)

To further investigate the function of the Pgt-IaaM gene in pathogenesis, 4-week-old 35S: Pgt-IaaM transgenic Arabidopsis and Col-0 untransformed plants were challenged with a low level of Psi DC3000 inoculum (10⁴ CFU/mL). Four days after inoculation, wild type Col-0 leaves developed chlorotic lesions with mild necrosis, whereas Pst DC3000 inoculation caused more severe yellowing and necrosis in leaves of all three transgenic lines (FIG. 8A). The growth of Psi DC3000 inside the inoculated leaves was also measured. Pst DC3000 multiplied 10 times more inside the inoculated leaves of transgenic lines than the Col-0 plants (FIG. 8B). Thus, the elevated growth of Psi DC3000 in the transgenic lines was consistent with the observed disease phenotypes. Together, these data demonstrate that expression of Pgt-IaaM in Arabidopsis enhanced virulence of Pst DC3000 growing on Arabidopsis.

### Discussion

Proteins with haustoria-specific expression in rusts and other biotrophic fungi are considered likely candidates for important effectors because of the importance of these cells in acquiring nutrients and altering their host cell environment Haustoria specific expression of a gene encoding a protein involved in a hormone biosynthetic pathway indicates Puccinia rust fungi make plant hormones as effectors. The Pgt-IaaM gene showed haustoria-specific expression. The experiments demonstrating that transient silencing of the gene reduced virulence indicates that production of IAM is an important component of pathogenicity in P. graminis and possibly the other Puccinia species.

The enzyme encoded by the IaaM genes makes indole-3-acetamide from Tryptophan. The Pgt-IaaM gene enhanced IAM and IAA production when expressed in Arabidopsis and conferred a phenotype characteristic of auxin overproduction indicating the gene codes for a functional enzyme. A second enzyme, indole-3-acetamide hydrolase, encoded by an IaaH gene, is used by many microbes and possibly plants to make IAA. In plant pathogenic bacteria and several fungi that make IAA via the IAM pathway, both genes are generally present in the genome. The three Puccinia species for which genome sequence is now available are unusual in that only the gene encoding the first enzyme is present. This raises the question of how IAA production might be boosted with just the addition of one of the two enzymatic steps by the fungus. However, genes encoding proteins with indole-3-acetamide hydrolase activity have been identified in Tobacco (NtAMI1 gene) and homologs are present in many species, including wheat. Alternative pathways may also be used to make IAA from IAM in Arabidopsis. In wheat, production of IAM may be rate limiting for IAA production by the IAM pathway, or any other pathways that utilize IAM, is but this limitation is relieved in cells containing Puccinia rust haustoria.

While most rust effector proteins are thought to be secreted from haustorial cells, it is not known where the IAA is actually synthesized in wheat cells harboring haustoria. The predicted protein sequences coded by the Pgt-IaaM gene had no apparent signal peptide to direct secretion of the protein. This suggests the IAM is made in the haustoria and possibly then enters the plant cell cytoplasm where the plant AMI1 protein is located (Pollmann et al. 2006) to make IAA. Small amounts of IAA were detected in Pgt uredinial spores, indicating that Pgt can synthesize IAA. This is likely synthesized via another IAA biosynthetic pathway. Many fungi, including biotrophs and saprophytes, make IAA but multiple pathways are used.

Plant pathogens enhance susceptibility to host plants by modulating the hormonal balance of the plant cells. However, relatively little is known about hormone involvement in the interactions between plants and cereal rust fungi. In the Puccinia-wheat system, small amounts of IAA was detected in plants at 2 days after stem rust infection, and then free IAA levels noticeably increased by 4 dpi as the infections spread through the plant tissues and the free IAA levels increased to approximately 5 ng/g FW by 6 dpi. The low levels of IAA early in infection may be partly because relatively few haustoria have been established, but it is also possible that the initial slight increase of local IAA concentration stimulates the host plants to further amplify the auxin biosynthesis pathway. The demonstration of the functionality of the Pgt-IaaM gene and haustoria specific expression indicates that the fungus is modulating the increase of IAA in the host cells and a synergistic effect between Pgt-and plant-derived auxin may exist. The observed increased bacterial susceptibility in Arabidopsis expressing Pgt-IaaM along with previous work showing susceptibility linked to auxin overproduction agrees with this interpretation. How localized increases in auxin makes the plant more susceptible is open to question. Previous work showing that pretreating rice plants with IAA increased susceptibility to bacterial and fungal pathogens associated the treatment with loosening of the cell wall, the natural protective barrier of plant cells to invaders. It remains to be seen whether cell wall loosening occurs in plant cells associated with rust haustoria.

ABA levels were also increased in Pgt challenged wheat tissues in a similar pattern to IAA. Alternatively, levels of the cytokinin trans-zeatin dropped sharply. Biosynthetic pathways for these compounds in plants are complicated so it is not clear if these concentration changes are directly mediated by the rust pathogen or a plant response to infection. The observed increases in ABA and decreases in cytokinin are consistent with the idea that these changes would benefit the pathogen and could conceivably be directly mediated by it.

BSMV-mediated HIGS has emerged as a powerful tool to study the functions and importance of candidate genes from biotrophic fungi because it enables knockdown of expression of target genes without stable transformation of the pathogen or host. The present study found that the HIGS phenomenon works in P. graminis and identified a target gene, Pgt-IaaM, as essential to full pathogenicity. The IaaM genes in the three Puccinia species are highly conserved (˜80% identical in amino acids) and lie on partially syntenic chromosomal regions, indicating they were present in their genomes before the three species diverged and that they represent an ancient strategy for pathogenicity. Blocking the action of one of the more conserved pathogenicity mechanisms in transgenic cereals may have great potential for engineering durable resistance to multiple rust diseases in cereals.

### Materials and Methods

### Plants Materials, Fungal Races, and Growth Conditions

The plants used in this study included wheat cultivar McNair 701, the Sr31/6*LMPG that carries the Sr31 gene and Arabidopsis thaliana Col-0. McNair 701 was used for stem rust Pgt gene-silencing assays, Sr31/6*LMPG was used as a stem rust resistant control and Col-0 was used as a source of wild-type Arabidopsis for transformation experiments. Wheat seedlings for gene-silencing assays were sown in pots containing potting mix and placed in growth chambers as previously described (Yin et al. 2011). Arabidopsis seeds were surface sterilized and cold-treated as described in Sandhu et al. (2012) and sown on ½× Murashige and Skoog (MS) medium (PhytoTechnology Laboratories Inc., Shawnee Mission, Kans.) containing 0.8% (w/v) phytablend (Caisson Laboratories Inc., Rexburg, Id.), 1.5% (w/v) sucrose, with appropriate antibiotics (30 μg/ml kanamycin). Germinating Arabidopsis seeds were incubated in darkness at 4° C. for 4 days, and then transferred to incubators with constant white light (30 μmol/m²/sec) for one week at 25° C. For observation of root phenotypes and etiolated hypocotyls, 1% (w/v) PHYTAGEL™ (Sigma-Aldrich, St. Louis, Mo.) plates were used in a vertical position. Arabidopsis seedlings were then transplanted to pots containing potting mix and placed in growth chambers with white light (200 μmol/m²/sec) set at 21° C. and 60-70% humidity. Approximately 100 mg of tissue from different developmental stages (10-day-old seedlings, 4-week-old rosette leaves, 6-week-old cauline leaves, and flowers) of Col-0 and transgenic Arabidopsis plants were harvested and stored at −80° C. for further use. Urediniospores of P. graminis strain CRL 75-36-700-3, race SCCL (Pgt7A) were increased on McNair 701 as previously described (Yin et al. 2011). Wheat leaves in different rust-infected stages (2 days, 4 days, and 6 days) and healthy plants were harvested for hormone measurement. Fresh spores were collected and used in inoculation experiments or stored at −80 ° C. for RNA or hormone extraction.

### Construction of BSMV-Derived Vector, in vitro Transcription of Viral RNAs, Virus and Rust Inoculations, and Rust Disease Assays

BSMVγ: Pgt-IaaM was constructed and viral RNAs were synthesized in vitro as previously described (Yin et al. 2011). In brief, 179 by of coding sequence of Pgt-IaaM was amplified from cDNA of rust infected wheat leaves using primers: 5′-ATAAGAATGCGGCCGCTAAACTATCAAGTCTTGGAGCATTCACTCTGG-3 (SEQ ID NO: 35) and 5-CCTTAATTAAGGGACATTCATGGAAGTCCTCAACGC-3 (SEQ ID NO: 36). The amplicons were double digested with NotI and PacI and directionally ligated into NotI/PacI sites of the BSMV γ vector. The derived pγ construct, pα, and pβ Δβa were linearized by BssHII, MIuI, or SpeI digestion, respectively. In vitro transcripts were prepared from the three linearized plasmids using the mMessage mMachine® T7 in vitro transcription kit (Ambion, Austin, Tex., U.S.A.) following the manufacturer's instructions. First and second fully expand leaves of 12-day-old wheat cultivar McNair 701 plants were inoculated with the transcripts produced from the BSMV construct carrying the Pgt-IaaM gene fragment. By 10 dpi, when virus symptoms became apparent on newer uninoculated leaves, only those leaves displaying mild virus symptoms were inoculated with Pgt7A spores. The infected leaves were harvested for RNA extraction at 5 dpi. The other infected plants were kept in the growth chamber until 12 dpi, and the infection types were assessed based on a 0-to-4 rating scale (Stakman et al. 1962). The BSMV: MCS construct was used as negative control. At least three independent experiments were conducted.

### RT-qPCR and RT-PCR Analysis

RT-qPCR analysis was performed as described in Yin et al. (2009; 2011) to estimate Pgt gene expression in different developmental stages (urediniospores, infected leaves, and purified haustoria) and also to estimate levels of gene expression after HIGS. To evaluate gene expression in different developmental stages, fresh urediniospores were collected from infected leaves at 12-14 dpi; infected leaves were harvested at 5 dpi and haustoria were isolated from infected leaves at 5 dpi. Three biologically independent samples were used for each developmental stage. To evaluate the extent of gene silencing, the infected wheat leaves challenged with virus and rust were harvested at 5 days after rust inoculation. Six biological replications were included for both the BSMV construct and the BSMV vector control constructs. The ratios of expression of each putative silenced seedling leaf were compared with each of the BSMV control seedlings, typically giving six estimates of silencing for each seedling. RT-qPCR was performed using Pgt-actin transcript to normalize the amount of cDNA in each of the samples, which was amplified with the primers 5′-TGTCGGGTGGAACGACCATGTATT-3′ (SEQ ID NO: 37) and 5′-AGCCAAGATAGAACCACCGATCCA-3′ (SEQ ID NO: 38).

RT-PCR was conducted to measure target gene expression levels in Arabidopsis transgenic lines. Total RNA was isolated from 4-week-old plant leaves. The Actin 2 gene (At3g18780) was used as an internal control to normalize the amount of cDNA in each of the samples, which was amplified using the primers 5′-GACCTTTAACTCTCCCGCTATG-3′ (SEQ ID NO: 39) and 5′-GAGACACACCATCACCAGAAT-3′ (SEQ ID NO: 40) in amplification reactions for 22 cycles. The Pgt-IaaM gene was amplified for 30 cycles using the following primers: 5′-GGGCAACAAGAATGGGAAGA-3′ (SEQ ID NO: 41) and 5′-CCACTAAGCGGCAGATGTAAG-3′ (SEQ ID NO: 42). All reactions were repeated three times with consistent results.

### Expression of Pgt-IaaM in Arabidopsis

To generate the Pgt-IaaM expression construct, the coding region of Pgt-IaaM was amplified from cDNA of rust infected wheat leaves by PCR using following primers: 5′-GCGTCGACATGAACTCCGTCAACTACCAAG-3′ (SEQ ID NO: 43) and 5′-AACTGCAG CATACAGTCATCTTTGAACACCAC-3′ (SEQ ID NO: 44). The PCR product (1790 bp) was digested with SalI and PstI and ligated into binary vector pCHF3 with the same restriction enzymes as previously described (Neff et al. 1999). The derived construct was electroporated into Agrobacterium tumefaciens strain GV3101. The A. tumefaciens strain carrying Pgt-IaaM was transformed into Col-0 according to the floral dip method (Clough and Bent 1998). Multiple transformants were identified by screening on plates containing 30 μg/ml kanamycin. Three representative lines with high levels of Pgt-IaaM expression (based on RT-PCR analysis described above) were chosen for further analysis.

### Roots and Hypocotyl Length of Arabidopsis Measurements

To measure hypocotyl length, 5-day-old seedlings grown on ½ MS media with 1% (w/v) phytagel were removed from plates and placed on transparent sheets. The seedlings were digitized with a flatbed scanner at a resolution of 600 dpi. The hypocotyls and root were measured from the scanned images that included a 1 mm scaled ruler and ImageJ 1.29J (National Institutes of Health, Bethesda, Md.; see the website located at rsb.info.nih.gov/ij/java1.3.1). All experiments were done in triplicate (n≧50).

### Hormone Measurements

The plant materials (described above) and urediniospores (approximately 100 mg fresh weight) were placed in 1.7 ml microcentrifuge tubes and extracted in 1.0 ml of Bieleski solvent (methanol:chloroform:formic acid:water (12:5:2:1)) using a TissueLyser II (Qiagen, Valencia, Calif.) at a frequency of 27 Hz for 3 min after adding two 2.8 mm diameter steel balls. The tube content was ultrasonicated for 3 min and then stirred for 10 min at 4° C. After centrifugation (10 min, 15,000 rpm, 4° C.) the supernatants were lyophilized. The dried extracts were dissolved in 50 μl of mobile phase (acetonitrile:water (5:95), 0.1% formic acid) prior to UPLC-MS/MS analyses. Plant hormones were measured with a UPLC-ESI-qMS/MS (ACQUITY UPLC System/XEVO TQ, Waters, Milford, Mass., USA) with an HSS column (ACQUITY UPLC HSS T3, 1.8 μm, 2.1×100 mm, Waters). Plant hormones were separated at a flow rate of 0.3 ml.min⁻¹ with linear gradients of solvent A (0.1% formic acid) and solvent B (0.1% formic acid in acetonitrile) set according to the following profile: 0 min, 95% A; 0.5 min, 95% A; 7.0 min, 50% A; 7.5 min, 5% A; 10 min, 5% A; 10.5 min, 95% A; 13 min, 95% A. Capillary voltage was 2.5 kV. Selective multiple reaction monitoring (MRM) mode using mass-to-charge (m/z) transitions of precursor and product ions was performed (m/z 176.1→130.0 for indole-3-acetic acid (IAA), 175.1→103.1 for indole-3-acetamide (IAM), 265.2→135.0 for abscisic acid (ABA) and 220.1→136.1 for trans-Zeatin (tZ)). Cone voltage (V) and collision energy (eV) were as follows: IAA: 18 V, 24 eV; IAM: 18 V, 26 eV; ABA: 28 V, 12 eV; tZ: 34 V, 20 eV. Data were processed by MassLynx™ software with TargetLynx™ (version 4.1, Waters). Stable isotope-labeled standard compounds were purchased from OlChemim Ltd. (Olomouc, Czech Republic) and standard compounds were purchased from Sigma (St. Louis, Mo., USA).

### Bacterial Growth and Disease Assays on Transgenic Arabidopsis

Pseudomonas syringae pv tomato DC3000 were grown on KB medium containing rifampicin (50 μg/ml) and ampicillin (50 μg/ml) overnight at 28° C. Cultures were centrifuged at 5000×g for 10 min, and bacterial pellets were washed twice with sterile ddH₂O and re-suspended in 10 mM MgCl₂ for plant inoculations. The bacterial disease assays were performed as previously described (Xiao et al., 2007). In brief, leaves of 4-week-old Arabidopsis thaliana transgenic plants were infiltrated with P. syringae DC3000 at 10⁴ CFU/mL using a needleless syringe, and bacterial growth was monitored at 0 and 4 dpi using serial dilution plating of ground leaf disks. Six leaf discs (0.5 cm² each) from three inoculated plants were collected with a cork borer. Two discs were ground in 1 mL of sterile water, diluted to the desired concentration, and plated on TSA medium containing 50 μg/ml rifampicin and 50 μg/ml ampicillin, and then cultured at 28° C. for 48 h and cell numbers were counted. Disease symptoms on Arabidopsis leaves were photographed 4 days after inoculation. Three independent experiments with three biological repetitions each were conducted.

### Statistical Analysis

Data analyses were performed using the general linear models (GLM) procedures on SAS statistical software (SAS Institute, Inc., Cary, N.C.). Multiple comparisons were performed by the Tukey's test or t-test (p≦0.05). Significance was accepted at α=0.05.

### REFERENCES

Clough, S. J., and Bent, A. F. 1998. Floral dip: a simplified method for Agrobacterium-mediated transformation of Arabidopsis thaliana. Plant J. 16: 735-743.

Line, R. F., and Qayoum, A. 1992. Virulence, aggressiveness, evolution, and distribution of races of Puccinia striiformis (the cause of stripe rust of wheat) in North America, 1968-87. In: Tech. Bull. No. 1788. United States Department of Agriculture, Agricultural Research Service, Washington, D.C.

Sandhu, K. S., Hagely, K., and Neff, M. M. 2012. Genetic interactions between brassinosteroid-inactivating P450s and photomorphogenic photoreceptors in Arabidopsis thallana. G3 (Bethesda). 2: 1585-1593.

Stakman, E. C., Stewart, D. M., and Loegering, W. Q. 1962. Identification of physiologic races of Puccinia graminis var. tritici. Agric. Res. Serv. E 617. U. S. Dep. Agric. Washington, D.C.

Xiao, Y., Lan, L., Yin, C., Deng, X., Baker, D., Zhou, J. M., and Tang, X. 2007. Two-component sensor RhpS promotes induction of Pseudomonas syringae type III secretion system by repressing negative regulator RhpR. Mol. Plant Microbe Interact. 20: 223-234.Yin, C., Chen, X., Wang, X., Han Q. M., Kang, Z., and Hulbert, S. H. 2009. Generation and analysis of expression sequence tags from haustoria of the wheat stripe rust fungus Puccinia striiformis f. sp. tritici. BMC Genomics 10: 626.

Yin, C., Jurgenson, J. E, and Hulbert, S. H. 2011. Development of a host-induced RNAi system in the wheat stripe rust fungus Puccinia striiformis f. sp. tritici. Mol. Plant Microbe Interact. 24: 554-561.

### Example 3

### Stable Transformation of True Grass to Silence a Pathogen Gene of Interest

To make plant cultivars that are resistant to rust fungi, one would first make a transformation construct that transcribes two copies of a DNA fragment of one of the rust gene sequences in opposite orientations. Upon transcription of this construct in a plant cell, a double stranded RNA of the fragment would be formed in the plant cell which would trigger the silencing of the fungal gene lithe fungus were present in that cell. The construct would carry a suitable promoter upstream of the rust gene fragments that would drive transcription of the fragments in plant cells. The promoter could be a constitutive promoter, like the maize ubiquitin promoter, or a promoter that was more specific to above ground, or photosynthetic tissues like leaves. A leaf-specific promoter would probably be adequate to confer resistance, but promoters that also functions in leaf sheaths and stems might provide better resistance to stem rusts which can infect these tissues in addition to leaves. The construct would also include a transcriptional terminator downstream of the fungal gene fragments. The construct would also carry sequences necessary for replication in bacteria and selectable markers for maintenance in the bacteria. The construct may also carry a gene(s) for selection in plant cells for the transformation process, like an antibiotic or herbicide resistance gene. Depending on the transformation method used, the construct may contain sequences necessary for replication in Agrobacterium and border sequences necessary for the Agrobacterium to direct the constructed DNA to the plant cell. Details of the construct would depend on the transformation system used to deliver it to plant cells. After the initial transformants were selected and cultured, they would be self-pollinated to provide T2 seed. T2 seed would then be planted and inoculated with the target rust species to identify resistant seedlings. DNA of these T2 seedlings would also be extracted and the presence of the construct in each seedling would be assayed by PCR or gel blot analysis. Families of T2 seedlings from several different transformants would be examined in this manner to identify families which had rust resistant seedlings when the construct was present. Rust resistant seedlings would be grown to maturity to produce seed for T3 families. T3 families would then be inoculated with rust to identify families where all the progeny seedlings were resistant to rust indicating they were homozygous and ‘true breeding’ for the construct and the rust resistance trait. T3 families with the best rust resistance and most stable expression between different family members would then be selected as a rust resistant transgenic line. This line would then be used in crosses with elite lines to begin a breeding program to transfer the rust resistant trait into commercial varieties.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A construct comprising one or more P. graminis f. sp. tritici (Pgt) genes selected from the group consisting of: PGTG_{—}11658, PGTG_{—}01136, PGTG_{—}03590, PGTG_{—}01215, PGTG_{—}03478, PGTG_{—}01304, PGTG_{—}07754, PGTG_{—}12890, PGTG_{—}14350 and PGTG_{—}16914.

2. A host plant that is stably transformed to contain and express fragments of one or more P. graminis f. sp. tritici (Pgt) genes selected from the group consisting of: PGTG_{—}11658, PGTG_{—}01136, PGTG_{—}03590, PGTG_{—}01215, PGTG_{—}03478, PGTG_{—}01304, PGTG_{—}07754, PGTG_{—}12890, PGTG_{—}14350 and PGTG_{—}16914.

3. A transgenic plant that is resistant to infection by a rust fungus, wherein expression of one or more pathogenic rust fungal genes is silenced by at least one heterologous nucleic acid in said transgenic plant.

4. The transgenic plant of claim 3, wherein said transgenic plant is a true grass.

5. The transgenic plant of claim 4, wherein true grass is selected from the group consisting of wheat, barley, sugar cane, and corn.

6. The transgenic plant of claim 3, wherein said rust fungus is a Puccinia species.

7. The transgenic plant of claim 3, wherein said rust fungus is a Puccinia fungus selected from the group consisting of P. graminis f. sp. tritici (Pgt), P. triticina (Pt), and P. striiformis f. sp. tritici (Pst).

8. The transgenic plant of claim 3, wherein said one or more pathogenic rust fungal genes are selected from the group consisting of P. graminis f. sp. tritici (Pgt) genes PGTG_{—}11658, PGTG_{—}01136, PGTG_{—}03590, PGTG_{—}01215, PGTG_{—}03478, PGTG_{—}01304, PGTG_{—}07754, PGTG_{—}12890, PGTG_{—}14350 and PGTG_{—}16914.

9. The transgenic plant of claim 3, wherein said transgenic plant is stably resistant to said infection by a rust fungus.

10. A method of making a transgenic plant that is resistant to infection by a rust fungus, comprising the step of
genetically engineering a plant to contain and express at least one heterologous nucleic acid that, when expressed in said plant, causes silencing of one or more pathogenic rust fungal genes in said plant.

11. The method of claim 10, wherein said transgenic plant is a true grass.

12. The method of claim 11, wherein true grass is selected from the group consisting of wheat, barley, sugar cane, and corn.

13. The method of claim 10, wherein said rust fungus is a Puccinia species.

14. The method of claim 10, wherein said rust fungus is a Puccinia fungus selected from the group consisting of P. graminis f. sp. tritici (Pgt), P. triticina (Pt), and P. striiformis f. sp. tritici (Psi).

15. The method of claim 10, wherein said one or more pathogenic rust fungal genes are selected from the group consisting of P. graminis f. sp. tritici (Pgt) genes PGTG_{—}1658, PGTG_{—}01136, PGTG_{—}03590, PGTG_{—}01215, PGTG_{——}03478, PGTG_{—}01304, PGTG_{—}07754, PGTG_{—}12890, PGTG_{—}14350 and PGTG_{—}16914.

16. The method of claim 3, wherein said transgenic plant is stably resistant to said infection by a rust fungus.


SEQUENCE LISTING (58 sequences)

SEQ ID NO: 1 - DNA, 48 bp, Artificial Sequence
  Synthetic oligonucleotide primer
ataagaatgcggccgctaaactatcaagtcttggagcattcactctgg

SEQ ID NO: 2 - DNA, 36 bp, Artificial Sequence
  Synthetic oligonucleotide primer
ccttaattaagggacattcatggaagtcctcaacgc

SEQ ID NO: 3 - DNA, 46 bp, Artificial Sequence
  Synthetic oligonucleotide primer
ataagaatgcggccgctaaactatgggacgtttattctgctttcag

SEQ ID NO: 4 - DNA, 33 bp, Artificial Sequence
  Synthetic oligonucleotide primer
ccttaattaaggtttccaaggagttcgggttgc

SEQ ID NO: 5 - DNA, 46 bp, Artificial Sequence
  Synthetic oligonucleotide primer
ataagaatgcggccgctaaactattgtttacggatcagccccagtt

SEQ ID NO: 6 - DNA, 34 bp, Artificial Sequence
  Synthetic oligonucleotide primer
ccttaattaaggaggtgttggtgtcctggttgaa

SEQ ID NO: 7 - DNA, 46 bp, Artificial Sequence
  Synthetic oligonucleotide primer
ataagaatgcggccgctaaactatcccttacggctaaaattgatgg

SEQ ID NO: 8 - DNA, 32 bp, Artificial Sequence
  Synthetic oligonucleotide primer
ccttaattaagggcattaccggggtattcgtg

SEQ ID NO: 9 - DNA, 46 bp, Artificial Sequence
  Synthetic oligonucleotide primer
ataagaatgcggccgctaaactatcgaatttttaggaccacaggcc

SEQ ID NO: 10 - DNA, 34 bp, Artificial Sequence
  Synthetic oligonucleotide primer
ccttaattaagggttgaatgccttgtaccttcca

SEQ ID NO: 11 - DNA, 44 bp, Artificial Sequence
  Synthetic oligonucleotide primer
ataagaatgcggccgctaaactataatccaaccaggctgcccca

SEQ ID NO: 12 - DNA, 32 bp, Artificial Sequence
  Synthetic oligonucleotide primer
ccttaattaaggcacgacaatcccgccgaacc

SEQ ID NO: 13 - DNA, 44 bp, Artificial Sequence
  Synthetic oligonucleotide primer
ataagaatgcggccgctaaactatagaactcttcccagtgccaa

SEQ ID NO: 14 - DNA, 32 bp, Artificial Sequence
  Synthetic oligonucleotide primer
ccttaattaaggatcccgtgtgccaagttaga

SEQ ID NO: 15 - DNA, 44 bp, Artificial Sequence
  Synthetic oligonucleotide primer
ataagaatgcggccgctaaactatatgcatcaggatcaggggag

SEQ ID NO: 16 - DNA, 32 bp, Artificial Sequence
  Synthetic oligonucleotide primer
ccttaattaaggactggggtttgtggaactga

SEQ ID NO: 17 - DNA, 46 bp, Artificial Sequence
  Synthetic oligonucleotide primer
ataagaatgcggccgctaaactataacttaagagactccgtcaacg

SEQ ID NO: 18 - DNA, 34 bp, Artificial Sequence
  Synthetic oligonucleotide primer
ccttaattaaggcgtgtcctggatgtatttgaca

SEQ ID NO: 19 - DNA, 46 bp, Artificial Sequence
  Synthetic oligonucleotide primer
ataagaatgcggccgctaaactatcatgacagtagctttgggagag

SEQ ID NO: 20 - DNA, 33 bp, Artificial Sequence
  Synthetic oligonucleotide primer
ccttaattaaggaatcctgtcgtgagtgggtgt

SEQ ID NO: 21 - DNA, 35 bp, Artificial Sequence
  Synthetic oligonucleotide primer
ccttaattaagggcaatccactaactgccaatcac

SEQ ID NO: 22 - DNA, 47 bp, Artificial Sequence
  Synthetic oligonucleotide primer
ataagaatgcggccgctaaactatcatggtgcgtagcgatgcaaata

SEQ ID NO: 23 - DNA, 34 bp, Artificial Sequence
  Synthetic oligonucleotide primer
ccttaattaaggatgggtggtttactcgaactcg

SEQ ID NO: 24 - DNA, 47 bp, Artificial Sequence
  Synthetic oligonucleotide primer
ataagaatgcggccgctaaactatgagcttctttgcacaatggtctg

SEQ ID NO: 25 - DNA, 36 bp, Artificial Sequence
  Synthetic oligonucleotide primer
ccttaattaagggaataccggaaatatgcacccgac

SEQ ID NO: 26 - DNA, 48 bp, Artificial Sequence
  Synthetic oligonucleotide primer
ataagaatgcggccgctaaactatctgtcaaaagtttggtggaaacgc

SEQ ID NO: 27 - DNA, 35 bp, Artificial Sequence
  Synthetic oligonucleotide primer
ccttaattaaggcactgagcctggcgataacactt

SEQ ID NO: 28 - DNA, 48 bp, Artificial Sequence
  Synthetic oligonucleotide primer
ataagaatgcggccgctaaactatcctcagatcccaatatcctgaagc

SEQ ID NO: 29 - DNA, 110 bp, Artificial Sequence
  Synthetic oligonucleotide primer
caccagcaacttttgcaaacataaaaatggtcgaccggtccccgcacccatacaagacctggtcaaggcaatctgctcgaaagccgcttcactaggggcaaccgtcagat

SEQ ID NO: 30 - DNA, 110 bp, Artificial Sequence
  Synthetic oligonucleotide primer
ataatccaaagtgtaaagctggaaagcctttgtatctgaaagtataactctgggatagttctctttgacctcttcattccaaaacttcttcactctggcaaaaaccttgg

SEQ ID NO: 31 - DNA, 110 bp, Artificial Sequence
  Synthetic oligonucleotide primer
caccgcatttggcaacgtccatggcgtgtacaagcctgggaatgtctccttgcagcccgaacttcttggcaagcaccaagcttactgcattctttgcaggcaagggtgtc

SEQ ID NO: 32 - DNA, 110 bp, Artificial Sequence
  Synthetic oligonucleotide primer
ttgagtgtcagtatcgacgttcatcttgaccacaccgttttcgagcgcagtcgcaatttccttcttggtggatccagatccaccgtggcggaatgcatgatcacggggat

SEQ ID NO: 33 - DNA, 27 bp, Artificial Sequence
  Synthetic oligonucleotide primer
caccgagagaaaagattggggtcaatc

SEQ ID NO: 34 - DNA, 21 bp, Artificial Sequence
  Synthetic oligonucleotide primer
tttgtggagtgggaggagacc

SEQ ID NO: 35 - DNA, 48 bp, Artificial Sequence; identical to SEQ ID NO: 1
  Synthetic oligonucleotide primer

SEQ ID NO: 36 - DNA, 36 bp, Artificial Sequence; identical to SEQ ID NO: 2
  Synthetic oligonucleotide primer

SEQ ID NO: 37 - DNA, 24 bp, Artificial Sequence
  Synthetic oligonucleotide primer
tgtcgggtggaacgaccatgtatt

SEQ ID NO: 38 - DNA, 24 bp, Artificial Sequence
  Synthetic oligonucleotide primer
agccaagatagaaccaccgatcca

SEQ ID NO: 39 - DNA, 22 bp, Artificial Sequence
  Synthetic oligonucleotide primer
gacctttaactctcccgctatg

SEQ ID NO: 40 - DNA, 21 bp, Artificial Sequence
  Synthetic oligonucleotide primer
gagacacaccatcaccagaat

SEQ ID NO: 41 - DNA, 20 bp, Artificial Sequence
  Synthetic oligonucleotide primer
gggcaacaagaatgggaaga

SEQ ID NO: 42 - DNA, 21 bp, Artificial Sequence
  Synthetic oligonucleotide primer
ccactaagcggcagatgtaag

SEQ ID NO: 43 - DNA, 30 bp, Artificial Sequence
  Synthetic oligonucleotide primer
gcgtcgacatgaactccgtcaactaccaag

SEQ ID NO: 44 - DNA, 32 bp, Artificial Sequence
  Synthetic oligonucleotide primer
aactgcagcatacagtcatctttgaacaccac

SEQ ID NO: 45 - DNA, 1767 bp, Puccinia graminis
atgaactccgtcaactaccaagcggaaataatagttaacatccacgagactgtgccccaagcttttattgataccccgagcattttatatcgcgaatggaatcagggaatcgaccagccacttggcactctcccaaaaaatctcaaaatttgtttgattggggcaggaatatcaaatgcgatagccgctcttgaactggtgaaagccggtgctcacgtcacactcctggaggctagatccgaggttggcgggagagcgcgatcggaaaaattcgaggatggcttcaatattgctgaaatgggcgctatgcgattcccgattaccagtggacttttactatattacgcgaaggaattcggttactgctttatttcagatttcccagacccgggtagggttccaacttttatttcttatagaggccgaaatgtctactggaagaatctcactgcggcccccaacggtttcgagaaggtgcatgtgggttgggatacgtttgtaaaaaatggattaaaaaatgacgacacagagatttttacatctctatatcatttaagaactttgcttcaatcgaaagatcaagaagtgaggctcacagcagttcctctgtggcagaaatacttggatttcttcaagaaagacagctttttcggggggcttcgaaggatttttggacctgagcacgagtgggacatccccgggcaacaagaatgggaagaaaatgattttgaaatgttcggaagtctaggaataggttgcggcggctatgcacctgttttttgcagagactttacttacatctgccgcttagtggtcaatggactatcaagcgagcaggccacttttgcaaaaatgaaaaacgatcaaccatttcccgcacccatacaagaattagtcaaagatatttgctcgaaggcggcctcactaggggcaaccgtcagattggaaacacgcggcgaagtagtttcttcttgcaacatcaacggctcttcctcagtaacggtggaggaaacttgccatggcgtcacaacggagaatgtttacgaccttgtcatcattggcactactacccgcgctgcaaacgtgatgatgagatatctcagtagttattcctccaaaatcctttctgaagctgccagcacggcagtaaacaacatcagcatgatttcttcaaccaaggtttttgccagagtgaagaaattttggaatgagaagagcaaagagaattatccaagagtcatcttatcagataccaaagcttcccagctttatactttggactacggacatccagattatgcaatagttttgctgacatatgcctggggtgatctatcagatcagatcatgacagtcaaaaacgataaggaactctatcgaattttgaaaaatcagatccatgagatcatgcgcgactccgacttcagtgactatgcagaacaattaactcccgtcgaagaaaacgacatacgtgtagttcactggcaacttgatccaacaagtcttggagcattcactctggccaggcctggccaagcgcgattgattgctgacgcattttacgacttcaaaaggctattggaagaccaagatcactccggagttttacttaatggtgattctatcaccttcgaaggaggatggattgaggctgcgttgaggacttccatgaatgtcacatcagcaatcctgctcaagtatggatctgtagaaggatatagtaaggcaccaataaacttgctagatagacatatttataagtattag

SEQ ID NO: 46 - DNA, 1080 bp, Puccinia graminis
atgggtggtctactcgaactcgtacctgctggtgtcgtcactggcaagaatctcgtcaaagtcttggagtatgctaaggctcacggattcgccattcctgccttcaattgcacatcttcttcgaccgtcattgcggccttagaaggagcccgagactccaaagcacccgtgatgattcaagtctctcagggtggtgcggcattctttgcaggcaagggtgtcaaaaacggtaaccaggaagcttcgattgctggtgccgtagccgctgccatgtttgtcagatccatcgctcccacttacggaatccccgtgatcatgcattccgatcattgtgccaagaaactgttgccctggtttgatggaatgctcgcggccaatgaggagtactacaagacccataaggaacccttattctcgtctcacatgctcgacctttccgaggaatctaaggaagaaaacatcgagatctgttgcaagtacttcgaaagaatggccaagattgatctctggctagagatggaaatcgggatcaccggtggtgaggaggatggcgttaacaatgaaaacgttgataatgctgcgttgtacacccaacccgaggatatttgggacgtttattctgctttcagcaagatcagcccgatgttttcgattgcggctgcctttggcaacgtccatggggtatacaaacctggaaacgtctctttgcaacccgaactccttggaaaacatcaagcttactgcaaggagaaacttaagaccgacgacccacgacctctctatctggttttccacggtggatctgggtccacgaagaaggaaatcgcgactgcgctcgaaaacggtgttgtcaagatgaatgtcgacactgacactcaatgggcctacatggaaggtttccgaaactacttcgattccaagaaggattatctgaaaactcaagttggtaacccggagggcgctgatcttcctaacaagaaatgctacgacccacgagtctgggtccgcgaaggagagaaaacgatggtcaagagagtcatggaagcttgccaagatctcaagaacgtcaacgtcctctaa

SEQ ID NO: 47 - DNA, 387 bp, Puccinia graminis
atgagaaccatttcaatcatgattacaattttctgtggcctgatctatcacgagacttatggagttaaggtgtctgaaattggatgtgatcgatggattaagggaagtaaagatgaagagagaaaagattggggtcaatcggtggtatatgtttacggatcagccccagttccgatctccgatagcggctgcgaacttacggtctcctcccactccacaaacaactggatatcccttttcaacggcgggcttgccatgcagtacctgctgctagatcacagaagcgggaagttttatgagtgtgcaattcaaccaggacaccaacacctagtggaagcagaaggctttgaaatcctggtcaggaaacttcaccgtagtgctaaatga

SEQ ID NO: 48 - DNA, 1056 bp, Puccinia graminis
atgggttcacaccttcttgcccctctccaacgaaaaatcattggcctgttttgcttagtcctgtttgctcaatccctagtagcggttcccgacgccagctaccacaactacaaaagtacacctaaaaactcgcaccaagttatcaagtcccgaggtcaaaatctaagacagactaaattttcatacttgggtccagccggtatcagagcccatgactcccttacggctaaaattgatggcgtctatattggcctgattccagatgccggagataccggcggcaatgtgcaaacgatgcgtgaaatcattaaatcgatcaacggcaaacgaccggcggtcttcggctggtatgcccaagtttcggttcataaagaatttgatggtagccagctgttcgaaatcctcgacgatttgaaaaaatcaaagtgtgtatttagtcccgccatcatgccccaaggtggctgggctggactaagacgcggtgacaatcatcaagcagttgccattgccaaggtccttcgaaagtttaccgatcaagagattccagtgatccttagatttgcccacgaggtcaattggtatcagcgtacgcacgaataccccggtaatgccgccgatttcaaggaggcttgggctgtccttgccgccgctattcgggagcacgctccagatgttcttcttcattggtgcccgaatgtatcaactatggaagagtaccgtaaatatgcacccgacaacatggactacacggtcgacatcgtcggatttgactactaccccaagagcaagccccaaccagacgaatttgtcaagaaggccaaagaattccacgatgcctttgccatcaacggcaggaaattcgccatgggagaagctggcctgcactaccaggcaagctacaaagaccgagtcgattggctttcacaaatggcccatgcccgaaaggaactccctaacttacttttcatcaactggtacaacagcaagaaggaatacgattacaagatcgcaggcaacggcctatccaccaccctcttgacagagaaactagaataa

SEQ ID NO: 49 - DNA, 1212 bp, Puccinia graminis
atgcgcggcccacatgtgatattcggaattggtctgtttttgaatattatacctctagcaaaggcacctgccttagacttgaacaacatcgatcaattgaagaaagcagcatctgcggcaatgagcaacctcatgtcttactacagtcccagttatagaggggcatttgacacggacatcacgccatggcaagattctttgatgatctggagtttgcattttgattatgccagtgatgttggagacacccgatacttggccaccgtccaaaatgcgctgtatcgtcaatcaaataatcagtcacactctttcctcgtcaagaatgtcgtcgaacaatggaatggtgggatcctctggggagctgaatggtcgttcaccctcacgcttgaagctgtggccgctgccgaatttttaggaccacaggccaagctacccaaatccagcgcaacctggataggtctcgcccagaaaaccttacaacaagtcgcgcctcagactgataatggctgtggagggggactcttttggtaccgaaatagggaagatcctaagcatggaaggtacaaggcattcaacactaacactgaattcatttcggccgctgctcgaagctttttgataacgaaagacacaaaggtcttaggattcgctcagcaacttgtagattggttgattgggccctcacgcctcgcagatgttaagactggaatcttacaggacggagtggatgccaaagacgcgagctgtcagctctctacggaccaatggacctacaactatggcctatggctgggctctatggcttggttgtttaaagcaacaggggatcaaaaatacctcaccatggcatcaaccgtttttactcactccgtcaagatctttgcccccaagggaattattgccgagatttgcgagccaacttcttgttcacgtgactccaaaggcttcaaggccgtctacatacgaaatctggtctacttacatcgagtgactaccgaccaggccatgaaaactgccatccagaatctcatcgatacctctgttaaagccatggcccaaacctcttgcgatgcaaagttcaactgtgccgctgcttgggctgcgggtagcccaccagagaaaaatgtccgctcacagcatgtgagcacagctttgttggtagcagctataggtatccgtaaacctcccgcaaacgctgccagagcaacatga

SEQ ID NO: 50 - DNA, 1011 bp, Puccinia graminis
atgtctcctcctgtagccaccgagtccatgtacaaacctactaccatcggcactgaagcccatgaccaagctttggctgccatgaaatccaaccaggctgccccagccaagccagtcttcaagccggaaccggccgtgaacttggagcctatcaagttcgctcctatcaaggagcaccaagtccaacgcgctatggtccgccgttacttccaagacatggaagagcgtgctatctctgacgtgatcattgtcggagccggttcggcgggattgtcgtgcgcctatgcgctcggcaaggcccgcccggatctcaagatcaccatcctcgagagcaatgttgctcctggcggtggctgttggttgggtggccaattgatgagtgccatggtgtgccgaaagcctgccgacaaattcttggatgaagtgggtgtaccctacgaggatgaaggtaacttcgtcgtcgtcaagcacgctgcgttgtttacctcgaccgtcttatccaaggtattggcgatgcccaacgtcaagatgtttaatgccaccgcatgcgaggacttgatcatcaagccttgcccaatcaacccgggcgtccaacgagttgctggttgtgtcaccaactggaccttagtctcccttaaccatgatcaccaatcatgcatggacccatcgaccatcactgccccacttgtctgctctttcgccggacacgacggtccatttggtgcgttctgcgtcaagcgtattgcctctgctggcttgagcgagggccttggagacatgcgtcccctcgacatggagcgtgctgaggaccacatcgccaacaagactcgtgaaatcgttcccggattgatcgtcggtggaatggaattgagcgagtttgacggaagtgctcgaatgggacctaccttcggcgccatgttactctctggtaagagagctgccgaagtggcgcttcaatctcttggtcgcgtcaaagtcgaagaaggtgaagttgttggctcggccaagtga

SEQ ID NO: 51 - DNA, 1179 bp, Puccinia graminis
atgtcaacaccgagcgatggagtgtaccaactgagtgtccaggatgatgcgactgatcgcaagttaacgctggacttgaccaggacggggttgacgatgcacaagctaacgatcggatctggtcgatttcctacccgggatgtgctctgtggacctgaatcaagttcaagcttcaaaatcgaaggaagaaagtttatgaatcaagtcgtcggaagatataccaatcgcttgccagccggtaagagttatataatcgattcctcgagggaggagggggggaagaggatagaaatagacttagacgagaatgaaccttttcaagatggacgagggaactgtttgcattctggacaaaatggctatgacttgagggagtttgagccagttggggccgattcaaaaatcgttcaagagtttcgatcgaccaacactgagcctgctaacagtgaatcagtatcaccgccatcattatcatcatcatctgcccattttcatctgcactctccttctggcgatcaaggcttcccagaggctatcgatatcctggctactgttaccttagaactcttcccagtgccaaaagataccgatctaactattccgagtgagaccggtcctaagagcaggatagggagagcaagttttagcctgagagcaaagttatcggacccagaggaggtcgcccgaagggactctggaacaccgatcaatctaacttggcacacgggatatatcctcaatgacttcagaggagagccgagtgactcgaatggaatccaacagcacaaattgtggatcaacagtgatcaccatctagcggtccatcaatcacaattgccaactggagaaatccagccaaacaaagcattggggatcgatttctcaactccagaggtcatcaggccaattggtgagatcatgcccccatcagggttcgatcagtgttttatattcaagaacacaactggagatacagtcgacaacactaaagacccaaaggcaatccttcgaagccctaaagacgacctgatcctggttttctattccaatcagacggcagtgcaatgttacagtgctaattatttcgatggaatcggttctcggaaagaacttcatcgacggactccggcagagcacgaagcaggtggatacaagaagcaaggcatgttatctttctaa

SEQ ID NO: 52 - DNA, 1521 bp, Puccinia graminis
atgcatcaggatcaggggaggaatacctatgtccaaatcgaatctcagtcagaatatgagcttcccagcgagttgcaggagccccaaaatccaaagacttgctcgagcaagatgaacaacttttttcaagctgctctatggtgtttggcaggtggtaccttgattggctttgcagtgtccgtcttgtggtgggaaccagtctgccagagtccaacgagctccgcttcaaactcagttccacaaaccccagtcctcaattacaataacacccaatcgagagtttattcctcaacacgagtcttcctcaaaaaagaagaggaatgtaacccatttcatgaaaaaggtttcctgcattacaatgctagtgagcccggcgataacacttggagaccttatgccccggattgtaaaccttccaacctttttcaaaaattgaaacaagaccttcattctactcgagagacttttggtgcaagtgagaaagacgaatttgcgtggttgaggaataggacggtggtgttgattggcgactcaatcgatagatggcataacgtcgatttctgtaacgtgctatcgtgggagccaggctcaacagagcgagaccccatcatatctgaaaatcgaccaaccaagacctactacgtcggtgcagactctccactatcaccccctccatggcactttcatccagaagatcctttgaaacctcctgaggattggcctgaggatgaacacgagctcttcaaatttcagacacccatttgggatgctggacaaatcaacgctaatacaaccagacctagggtttgcgaaattcctaaatacggattcagagtcgtcagtctctttagttggggcctggatccaaaggagggcggtagactttatgctaacatctctggctactttccacctgctgaatacacgagtcgaatcgaccacatcttgaagccaattttagaaaaccttgcaacgcaatccaacaacacgttagtcacgaagcccgacctcatagagatgtcatcaggcttctgggatcttcgacaatgggcagaagaggacaagaaaataatggagaaacggatcgggaaaccgattgattatcccactgaatatgggattggttttagcgatttggaccaggatcgattgaattggtggagcgaaagacagaagaaagctatccgattcgtctccgacgcttttcctcaatcccaaactcctatcctctggcgaagcttgcatcacactcagacccatcattgggtcgggtacgatcgagtcaaccagatcgatcaattggctagatataatatcgagatgttgaaactggaagattctagtctcgagccccgcctgaggattgatgaatggggtgctcttatgcttggtcaagaacaccatttcagggacgtgatgcatgtccgcgaactgcctggtggagtcttatggggagacgtgatgctttgggaacttcgacgtgcagtcaagaaacgtggatcaacttattactcagcgtga

SEQ ID NO: 53 - DNA, 1020 bp, Puccinia graminis
atgcaccaccactccaacttcctccccacccactctaaagtcctcctcctcctcctcctcctcttcatcaccctccacaccccaaacaccgcgatggctgctaccaagaagcttctagtgggctatgtccccgagcatttctcggcaccgctactccagctgctcgagcacgacaaggagctcgcagaatgggtcgagctggtgcccaacccgagcgggaccggacagatgatcagcgccctcaaggagcgctccatccatctcgccgtcgcactcaccgagtcgctcatcgccgggatcatcctcggcaaaggggactacaaactcatcggcacctatgtcaccagccccttgaactgggccgtcatcactggggctaacaccaagtacaaatcgatcgccgacttgaaaggccaaaagatgggaatcagtcgtccaggctctggctcacaagtgatggcctccgtcatggcgctccaacaaggctgggctaactctcccgactcggccctcgaatttgtcgtgaaagactcgtttaaaaacttaagagactccgtcaacgacggctccactgcggccttcatgtgggaatggtttacaactaagccttaccaggactcgggagaagtcaagtttatcggcagccagcccacgccctgggaatcttggatgatcgctgcgtcccctgaatcggtcgatcccgagctcctggcgcccgagaagctgcggttattcttgagcaaactggacgaatcgatccacaggttcggggccgaactcgggatccgtagccccgaggatgtcaaatacatccaggacacgttccatcaaaaggaggaagacgttaaagaatggatgaaaggcgtcagttatccgaagaaaactcacgagatctctcagaagaccttgcaggaaaccgtcaaggatttgggactggccggagtgatcctggacgccagcaaggtcgtgatacctgaccaactcgtcaacactgatgtcgccaaggtgatccaaacctga

SEQ ID NO: 54 - DNA, 1674 bp, Puccinia graminis
atggagtccgaccacaaagggtttcaacatcggggcacttcctacgcagaatccgaaatgaaaaagcatgccgaccatggattttacgagacgaaagaagttaacgctgtgtcgtctactgccggtgatgttgcgattggcgagaacaccgtccaccgggctcttgagcagaggcacattagcatgattgcaattggaaccggactgttcttgggtatgggtgaggctctcaacaaaggaggacctcttgggcttgtgcttggatacttggtgatgggactcgttgtttatgccatgacagtagctttgggagagatggtgacaatgttcccggtctctgggtcatttactcactacccatgtcgtttcgtagacccagcgcttggcttcgccgttggatggaattactggtattcgatgacaatcgccgtcccgagtgaggttatcgcatccgccattgtcgtggagtattggaaaactcccgtgaataaagcagtttgggtgacactgttcattgtactcggatgtgcgatcaacttcctcggggtgagatggtacggagagacagaattcgcgttcgccgcggtaaaggtcgtcgcggtggtcatcctgattctagtcggaatagttattgatttaggcgggacacccactcacgacaggattggacttcggaattttattaatcccggaccattcaaccaattacatgagataccaggagccgttggcaggttcttagctttctggacggtatttctccaagcaacttactcttacatgggaacagaaatggtcgcaatcacagccggcgaagccgccaatcctcgaaaaaccgtacccaaggccattgaacgagtcttttaccgaatcttggttttctatttgggaagcacccttaccgtcggtcttttggtcccttcctcatccccggatctcttggggggtactggtgatgccagctcttcgccattcgtaattgcaatcaaagctgctggaatcaccgtacttcctgatttgatcaatgtcgtcattctggtatcagcgatttcggccgcaagctctaagctttacggtggtagccgagttttgttcggcctttcaaaagatggaatggcacctcgaatattctctaagtgcaacggttctgggctacccatatgggcattaatggcaacttgtagtgctgctggtctctcttacatgtgcctgaataacaaagcgagtcttgccttccaatggttccagaatctctcagctatgactggtatcttgacgtggtgggcagttttagtatcctacctatttttttacaatggcttgaaagtccagggttatagtcgagacgcattacactataaagctccatttcaaccatatgcttcctggataggcgtgattatgctcacgctcattatgttcatgagcggcttcgagacttttctgaaaggtgactggtccacctcagattttctagcaagctacttgacactcccaattttcttgggcttctacctatactggaaaattaccaagcgaaccaaattcgtaagtcccaaacatatggactttgttactgggacacgtgagctagacgaaatggatgcactggagagagaaaaggcagtcgttcccaccacgaaatggggcaaattcattgactggttaatgtaa

SEQ ID NO: 55 - DNA, 1767 bp, Puccinia graminis
atgcaatccactaactgccaatcacaaatcatagctgaaatccatgagacaatccctcaagctttcattgataccccagctattttgtatcccgagtggaatcaggggatcgaacaatcactgggcacactacgaaatgaccttaaaatctgcttaatcggcggaggaatctcgaacgcgataactgcacttgagttggtaaaagcaggcgcccaagtcacgctcctggaggtaagagcagaggttggcgggagagcacgctccgaaacattcggtgatgggttcaacattgcggagatgggtgcaatgcgctttccaacatcaagtggacttttactttactatgcgaaggagttcggttactgctttatctcagacttcccagatccaggacgagtcccaacgttcatttcttatagaggccagaatgttttctggaagacactcagcgcgtccccaaacggcttcgagaaggtacacgttggttgggatacttttgtaaaagatggattgaaaacaaacggagtcgaggtatttgcatcgctacgcaccatgcgaattttactccaatctggagaccaagaagtgagacaagcagtgattcctctatggcagaaatttctggattccttcaagaacgacagtttcggcgggggactccaaaggatatttggcccgcagcacaattgggacgttcctgggcggcatgtatgggaagagcaagatttcgaaatgtttgggagtttgggaataggatgtggcggttatggtcctgtctattcgagagatttcaattccatccttcgcctggtagttaacggattagagagtgatcaagccacttttgcaaagatgaaggatggtcgaccattacccgcacccatccaggatttggtcaaggagatttgcttgaaggcttctagcttgggggcaacaatcaaactgaaaaccaatggggaagtcatttcctctcacagcatcaacggcgctccctcaataattgtggaggaaacttgtgacgacgtaacaaatgagagtaattacgacttggttataattggaaacactacgcgtgctgcaaacgtgaagatgagatatataagtagttattccaacaaaatcctctccaaggctgccagcactgccgtgaacaacattagcatgatttcttcaaccaaggtttttgccagagtgaagaagttttggaatgaagaaaacaaagagaactatcccagagttatactttcagatacaaaggcttcccagctttacactttggattatggacatcctgaatatggaatggtactgctcacatatgcttggggggacctttcggatcagattatgacagtcaaagacgataaggaactctatcgaatcttgaagaatcagattcacgaaattatgcgtaattcagagtttgccgactatgcagaacagttggctcccgtcgaagagaacgacattcgtgtggttcactggcaacttgatccaacaactcttggtgcattcgctctggctatgccaggtcaagcccggatgatcgcagacgctttttatgacttcaaaagcttatcggaggacaaagatcattcaggagtgctactgaatggtgattcaatcacctttgaaggtggttgggtagagggtgctttgagaactgcgatgaatgtcacatccgcgatcatgcttaagtatggatccgtggaacaagaaagtaaggcacccgtgaacttgctcgaaagaaatacttacaagtattag

SEQ ID NO: 56 - DNA, 1176 bp, Puccinia graminis
atgggtggtttactcgaactcgtacctgtgagtttcgatttactagcactagataagattgaaccctgtcttacccacccatactaccccctatcttcatcatcgcttggtaacctaggcaacagtggtgtcgttaccggcaaaaaccttgtcaaagtcttggagtatgccaaagccaaaggattcgctatccctgccttcaactgtacatcctcttcaactgttattgcggccttggagggagcccgagaatgtaaagccccggtgatgattcaagtctcccaaggtggtgcggcattcttcgcaggcaagggtatgaaaaatggcaaccaggaagcttcgattgctggggccgttgccgctgctatgtttgtccgatcgattgcacctgcctacggaatcccagtaatcatgcattcagaccattgtgcaaagaagctcttaccttggtttgatggaatgttaacagcaaatgaggagtactacaagaagcatggagaaccattattctcttctcatatgctcgatctttctgaggaatctaaggaagagaatatcgagatctgtgttaaatatttcgcacgaatggccaagatcgatctttggttagagatggaaatcggtatcactggtggtgaggaagatggcgttaacaacgaaaacgtcgataatgctgctctttacacccaacctgaagacatctgggacgtttactccgccttcagcaagattagccccatgttctcgattgcggctgcctttggtaacgtccatggtgtatacaaacctggaaatgtctcattacaacccgagctcctaggcaagcaccaagcttattgcaaagaacaacttaagacggatgacccccgacccctttacttggtcttccacggtggatctggatccaccaagaaggaaattgcgactgcgctcgaaaacggtgtggtcaagatgaacgtcgatactgacactcaatgggcttacatggaaggtttccgaaactacttcgactccaagaaagattacctgaaaactcaagttggtaacccagatgcacctgacggtcctaacaagaaatactacgacccacgagtttgggtacgtgagggagagaaaacaatggtcaagagagtcatcgaagcttgccgagatctcaaaaacgtcaacgtactttga

SEQ ID NO: 57 - DNA, 1071 bp, Puccinia graminis
atggactcaaacattctggcccccctcaatcgcaagatcctcggccttctctgcctcgccctcttcgcccaatccctacttgctgctcccgatgtaaattaccatggccatagagccacttccaaaaaccgtcaacactcccgagttgctcggaccgtcaggccccgaagtcaaaatatgagacaaaccaagttttcacacttgggacccgctgggatcagagcttatgactcacacacggccaaaatcaatggcgtctttatcggcctaattcccgacgctggagataccggcggtaacgtgcaaactatgcacgaaatcatccaatcgatcaacggcaaacgacctgcggtattcggatggtacgcccaggtttcggttcactcgaaattcgatggtagccaacttttcgaagtcatggacgatttgaagaaatcgaatgctgtattcagtcccgccatcatgccacaaggtggttgggcaggtcttagacgtggagataaccatcaagctgtcgccattgccaaggttcttcgacggttcaccgatgaaggggttccggtaattgcgagatttgcccacgaagtcaactggtatcaacgcacccacgaataccctggtgggcccgccgatttcaaggaagcatggggcgttcttgccgctgcagtccgggagcacgctccagatgtccttcttcattggtgcccaaatgtgtccacaatggaagaataccggaaatatgcacccgacaacatggacgaaactgtcgacctcgtcggattcgactactaccccaagagcaagcctcaagcaggcgagtttctcaagaaggccaaagaatttcacgacacctttgccatcaacggccggaagttcgcgatgggggaagcaggcttgcactaccaaggcacgatcgaagagcgagtcgactggcttttacaaatggccaactcccgaaaggaactcccgaacttgctgtacatcaactggtacaacagcaagaaggaattcgattacaagatcgcaggcaacggcgtttccaccaaacttttgacagaaaaattagaataa

SEQ ID NO: 58 - DNA, 1518 bp, Puccinia graminis
atgcatctggatgagggcagaaatacttatgtccaagtacaatctcaatcagaatatgagaactccaccgagctggagactcaaaccccaaagacttgctcgagccggatgaacaactttttccaagctgccttatggtgcttggcaggaagtactctgatcggctttgcaatttcggtactgtggtgggaaccagtgtgtcagatccccaagagctcgcattcaaccccagccttgacaaaaacagtccccaaccagaacaccacacaatcgaggatttactcttcaacaaggatgcttgtcaaacaggaagaggaatgtaatccatttcaagaaaaaggtttcctgcattacaacgccactgagcctggcgataacacttggagacctttcaatccggattgccaaccatccaacctttttgaaagattaaaagaagatctccattctcctcaagagtctttcggcgagagcacacgagacgagtttgcctggctgaggaatagaacagtggtattgattggcgactccatcgatagataccacaatttcgatttctgcaacgtgctctcgtggcaacccggatctacagaaggggagcccatcatatctgaaaaccgaccgacccggacgtactacattggtgcagactctccactttcaccccctccatggcacttccatcctgaagaccccttgaaacctcctgaagactggcctaaagacgaacacgaacttttcgcatatcagacaccgatctgggattccggaaagattaacgcaaataccactagacccagggtttgtgaaattcctaaatacggattcacgttaatcagtctttttaattggggcctagatccaaaagatggtggcaggctctatgcaaacatttcaggctatttcccgcctgctgaatacaccagccgagtggaccatatcctgaagccgatcctgcaaaacctggcaagccataataacaacacgatgatcaccaaaccagacttgatagagatggcttcaggatattgggatctgaggcaatgggcagaagaggacaagaaaatcgaggagaaaagacagagaaagaaaattttgtatccaagtcaagaaggaatcggtttcggtgacttggaccagactagattggattggtggaccgaaagacagaaaaaagcgatccgacatatctccaacgtttttcctgaacctcaaacccccatcctctggagaaccttacatcacacccaacctcatcattgggtcggctacgatagagtcaatcaaatcgatcaattggctagatataatgtcgagaagttgaaacgggaggaacccaatctggagccgcgcctaagaattgatgaatggggtgcccttatgcttggacaagagcaccactttcgcgatgttatgcatgtacgggaactgcctggtggcgtgttatggggagaccttatgctttgggaacttcgacgagcagtcaagaaacgtggatcaactcattacgcaccatga